SEQUENCE LISTING (19 sequences)

SEQ ID NO: 1 - DNA, 343 bp, Artificial Sequence
  Construct
aacttgtttattgcagcttataatggttacaaataaagcaatagcatcacaaatttcacaaataaagcatttttttcactgcattctagttgtggtttgtccaaactcatcaatgtatcttatcatgtctggatctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 2 - DNA, 4734 bp, Artificial Sequence
  Construct
aacttgtttattgcagcttataatggttacaaataaagcaatagcatcacaaatttcacaaataaagcatttttttcactgcattctagttgtggtttgtccaaactcatcaatgtatcttatcatgtctggatccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggcctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 3 - DNA, 26 bp, Artificial Sequence
  gRNA
cccggagtcgaagacagttctagggt

SEQ ID NO: 4 - DNA, 26 bp, Artificial Sequence
  gRNA
tcagtcttccaacggggccccggagt

SEQ ID NO: 5 - DNA, 26 bp, Artificial Sequence
  gRNA
tccggggccccgttggaagactgagt

SEQ ID NO: 6 - DNA, 26 bp, Artificial Sequence
  gRNA
agttcacaaccgctccgagcgtgggt

SEQ ID NO: 7 - DNA, 26 bp, Artificial Sequence
  gRNA
ccggccgctagggggcgggcccggat

SEQ ID NO: 8 - DNA, 26 bp, Artificial Sequence
  gRNA
agcggccggggagggaggggccgggt

SEQ ID NO: 9 - DNA, 26 bp, Artificial Sequence
  gRNA
cggccggcgaacggggctcgaagggt

SEQ ID NO: 10 - DNA, 26 bp, Artificial Sequence
  gRNA
ctcgaagggtccttgtagccgggaat

SEQ ID NO: 11 - DNA, 26 bp, Artificial Sequence
  gRNA
agctaagcgggtggcaaggggcgggt

SEQ ID NO: 12 - DNA, 26 bp, Artificial Sequence
  gRNA
ccccgcaaatgcgcagctaagcgggt

SEQ ID NO: 13 - DNA, 26 bp, Artificial Sequence
  gRNA
gctgggcccacggcaggagggcggat

SEQ ID NO: 14 - DNA, 26 bp, Artificial Sequence
  gRNA
ccgctaggaagcagccaatgacgagt

SEQ ID NO: 15 - DNA, 26 bp, Artificial Sequence
  gRNA
cagccaatgacgagttcggacgggat

SEQ ID NO: 16 - DNA, 26 bp, Artificial Sequence
  gRNA
tgttagtccactcgcacgcctcgaat

SEQ ID NO: 17 - DNA, 26 bp, Artificial Sequence
  gRNA
tcggacgggattcgaggcgtgcgagt

SEQ ID NO: 18 - DNA, 26 bp, Artificial Sequence
  gRNA
ggcgggggcggggcgcagggaagagt

SEQ ID NO: 19 - DNA, 26 bp, Artificial Sequence
  gRNA
cacctatgggcgtaggcggggcgagt

(19) United States
(12) Patent Application Publication
(10) Pub. No.: US 2023/0212538 A1
(43) Pub. Date: Jul. 6, 2023

(54) METHODS FOR INTEGRATING DNA INTO GENES WITH GAIN-OF-FUNCTION OR LOSS-OF-FUNCTION MUTATIONS

(71) Applicant: BLUEALLELE, LLC, Oakdale, MN (US)

(72) Inventor: Nicholas BALTES, Oakdale, MN (US)

(21) Appl. No.: 17/915,785

(22) PCT Filed: Mar. 29, 2021
(86) PCT No.: PCT/US2021/024725

§ 371 Date: Sep. 29, 2022

Related U.S. Application Data
(60) Provisional application No. 63/002,259, filed on Mar. 30, 2020.
(60) Provisional application No. 63/070,877, filed on Aug. 27, 2020.

(51) Int. Cl. C12N 9/22; C12N 15/11; C12N 15/86

(52) U.S. Cl. CPC C12N 9/22; C12N 15/11; C12N 15/86; C12N 2310/20; C12N 2800/80; C12N 2750/14143

(57) ABSTRACT

Methods and compositions for modifying the 3′ untranslated region or coding sequence of endogenous genes using rare-cutting endonucleases and donor molecules. The methods and compositions described herein can be used to modify the coding sequence of endogenous genes or to facilitate early termination of transcripts.

### SEQUENCE LISTING

The instant application contains a Sequence Listing which has been submitted in ASCII format via EFS-Web and is hereby incorporated herein by reference in its entirety. Said ASCII copy, created on Mar. 29, 2021, is named Sequence_Listing_1026008PCT.txt and is 10,168 bytes is size.

### TECHNICAL FIELD

The present document is in the field of genome editing. More specifically, this document relates to the targeted modification of endogenous genes using rare-cutting endonucleases.

### BACKGROUND

Monogenic disorders are caused by one or more mutations in a single gene, examples of which include sickle cell disease (hemoglobin-beta gene), cystic fibrosis (cystic fibrosis transmembrane conductance regulator gene), and Tay-Sachs disease (beta-hexosaminidase A gene). Monogenic disorders have been an interest for gene therapy, as replacement of the defective gene with a functional copy could provide therapeutic benefits. However, one bottleneck for generating effective therapies includes the size of the functional copy of the gene. Many delivery methods, including those that use viruses, have size limitations which hinder the delivery of large polynucleotides. Further, many genes have alternative splicing patterns resulting in a single gene coding for multiple proteins. Methods to correct partial regions of a defective gene may provide an alternative means to treat monogenic disorders.

### SUMMARY

Gene editing holds promise for correcting mutations found in genes that cause genetic disorders; however, many challenges remain for creating effective therapies for individual disorders, including those that are caused by gain-of-function mutations, or where precise repair is required. These challenges are seen with disorders such as myotonic dystrophy type 1 or spinocerebellar ataxia type 8, wherein the disorder is caused by expanded trinucleotide repeat sequences.

The methods described herein provide novel approaches for correcting gain-of-function or loss-of-function mutations. The disclosure herein is based at least in part on the design of bidirectional polynucleotides compatible with integration through multiple repair pathways. The polynucleotides described herein can be harbored on linear or circular polynucleotides and can be integrated into genes by the homologous recombination pathway, the non-homologous end joining pathway, or both the homologous recombination and non-homologous end joining pathway. Further, the outcome of integration in any case (HR, NHEJ forward, NHEJ reverse) can result in precise correction or alteration of the target gene's mRNA or protein product. The polynucleotides described herein can be used to prevent transcription downstream of the site of integration or they can be used to repair or modify the 3′ end of genes. The methods are particularly useful in cases where precise editing of genes is necessary. The methods described herein can be used for applied research (e.g., gene therapy) or basic research (e.g., creation of animal models, or understanding gene function).

In one aspect, the methods described herein provide novel approaches for correcting repeat expansion diseases (also known as microsatellite expansion diseases or trinucleotide repeat disorders). Repeat expansion diseases are frequently caused by an increase in the number of copies of a trinucleotide repeat within a gene, where the number of repeats crosses over a threshold where they become unstable. Generally, the larger the number of repeats, the greater severity of disease symptoms. Unstable trinucleotide repeats can result in a number of different consequences, including defects in protein function, changes in gene expression, production of toxic RNA, or increased chromosomal instability. Examples of repeat expansion diseases include myotonic dystrophy, spinocerebellar ataxia, juvenile myoclonic epilepsy, Friedreich's ataxia, and Huntington's disease.

In one aspect, the methods described herein are useful to correct repeat expansion diseases where the mutation results in production of toxic RNA transcripts, including myotonic dystrophy (DM) type 1, DM type 2 (DM2), fragile X tremor ataxia syndrome (FXTAS), SCA type 8 (SCA 8), SCA 10, SCA 12, SCA 31, SCA 36, Huntington disease-like 2 (HDL2) and amyotrophic lateral sclerosis (ALS). In one aspect, this document provides polynucleotides for integration into gene's comprising repeat expansion mutations. The polynucleotides comprise a first and second terminator in tail-to-tail orientation. When the polynucleotides are integrated upstream of a repeat expansion mutation, transcription from the endogenous gene's promoter will be terminated prior to the mutation. Integration by the NHEJ pathway in either the forward or reverse directions will result in termination of the transcript prior to the mutation.

The methods described herein are compatible with current in vivo delivery vehicles (e.g., adeno-associated virus vectors and lipid nanoparticles), and they address several challenges with achieving precise alteration of gene products for gain-of-function disorders.

This document features methods for integrating a polynucleotide into endogenous genes. The methods can include delivery of polynucleotides, where the polynucleotides are circular or linear, and harbor a first and second terminator in opposite directions. If the polynucleotides are linear, the polynucleotides can comprise a first and second terminator, and can be integrated in the 3′ UTR of endogenous genes, including the DMPK gene, ATXN8 gene and JPH3 gene. If the polynucleotides are circular, the polynucleotides can be linearized by a rare-cutting endonuclease prior to integration. The circular polynucleotides can be used to modify the 3′ end of an endogenous gene, or the 3′ untranslated region of an endogenous gene target. The polynucleotides described herein can be delivered on viral vectors (e.g., adeno-associated viral vectors) or by non-viral methods (e.g., lipid nanoparticles).

This document features a method of integrating a heterologous polynucleotide into an endogenous gene in the genome of a cell, where the method includes administering to a cell two recombinant nucleic acids. The first nucleic acid can include a sequence with, from 5′ to 3′, a first terminator and a second terminator in reverse complement direction. The second nucleic acid can encode a rare-cutting endonuclease targeted to a site within an endogenous gene in the genome of the cell. Following delivery of both nucleic acids, the first nucleic acid can be integrated into the endogenous gene to provide a modified endogenous gene in which the first terminator or the second terminator is operatively linked to a promoter of the endogenous gene, and wherein the modified endogenous gene produces an mRNA transcript that is truncated relative to an mRNA transcript produced by the endogenous gene. The method can include where the first nucleic acid is a linear double-stranded or a linear single-stranded DNA molecule. The method can further where the first nucleic acid is a circular double-stranded DNA molecule. The method can include where the first nucleic acid is a viral vector. The viral vector can be an adeno-associated virus vector, an adenovirus vector, or a lentivirus vector. The rare-cutting endonuclease can be used to facilitate integration of the first nucleic acid into the endogenous gene. The rare-cutting endonuclease can be a zinc-finger nuclease or a CRISPR nuclease. The method can include using a first nucleic acid that does not comprise a coding sequence and a coding sequence reverse complement operably linked to the first and second terminators. When the first recombinant nucleic acid is a circular double-stranded DNA molecule, the DNA molecule can further have a rare-cutting endonuclease target site 5′ of the first and second terminators (i.e., between the first and second terminators relative to the 5′ ends). The rare-cutting endonuclease target site within the circular DNA molecule can be the same target site as within the endogenous gene. The method can include integrating the first nucleic acid into the DMPK, ATXN8, ATXN8OS, or JPH3 gene. The method can include integrating the nucleic acid into the 3′ untranslated region of the DMPK gene. The method can include integrating the nucleic acid into the 3′ untranslated region of the DMPK gene downstream of the stop codon and upstream of the CTG repeat sequence. The method can further include using first nucleic acids with a left and right homology arm flanking the first and second terminators.

This document also features a method a method of integrating a heterologous polynucleotide into an endogenous gene in the genome of a cell, where the method includes administering to a cell a recombinant nucleic acid. The nucleic acid can include a sequence with, from 5′ to 3′, a first terminator, a sequence encoding a rare-cutting endonuclease targeted to a site within an endogenous gene in the genome of the cell, and a second terminator in reverse complement direction. Following administration to a cell, the nucleic acid can be integrated into the endogenous gene to provide a modified endogenous gene in which the first terminator or the second terminator is operatively linked to a promoter of the endogenous gene, and wherein the modified endogenous gene produces an mRNA transcript that is truncated relative to an mRNA transcript produced by the endogenous gene. The method can include where the nucleic acid is a linear double-stranded or a linear single-stranded DNA molecule. The method can further where the nucleic acid is a circular double-stranded DNA molecule. The method can include where the nucleic acid is a viral vector. The viral vector can be an adeno-associated virus vector, an adenovirus vector, or a lentivirus vector. The rare-cutting endonuclease can be used to facilitate integration of the first nucleic acid into the endogenous gene. The rare-cutting endonuclease sequence can be a zinc-finger nuclease coding sequence, a CRISPR nuclease coding sequence, or a gRNA sequence. The method can include using a nucleic acid that does not comprise a coding sequence and a coding sequence reverse complement operably linked to the first and second terminators. When the nucleic acid is a circular double-stranded DNA molecule, the DNA molecule can further have a rare-cutting endonuclease target site 5′ of the first and second terminators (i.e., between the first and second terminators relative to the 5′ ends). The rare-cutting endonuclease target site within the circular DNA molecule can be the same target site as within the endogenous gene. The method can include integrating the first nucleic acid into the DMPK, ATXN8, ATXN8OS, or JPH3 gene. The method can include integrating the nucleic acid into the 3′ untranslated region of the DMPK gene. The method can include integrating the nucleic acid into the 3′ untranslated region of the DMPK gene downstream of the stop codon and upstream of the CTG repeat sequence. The method can further include using first nucleic acids with a left and right homology arm flanking the first and second terminators.

This document features polynucleotides comprising a first and second terminator in a tail-to-tail orientation, wherein the polynucleotide does not comprise a coding sequence operably linked to the first and second terminators. The polynucleotides can be linear double-stranded or a linear single-stranded DNA molecules. The polynucleotides can be circular double-stranded DNA molecules. This document can feature a viral vector with the polynucleotide comprising a first and second terminator in a tail-to-tail orientation, wherein the polynucleotide does not comprise a coding sequence operably linked to the first and second terminators. The viral vector can be an adenovirus vector, an adeno-associated virus vector, or a lentivirus vector. The polynucleotides can further comprise an shRNA silencing cassette or a sequence encoding a rare-cutting endonuclease. The shRNA silencing cassette or sequence encoding a rare-cutting endonuclease can be positioned between the 3′ ends of the first and second terminators. When the polynucleotide is a circular double-stranded DNA molecule, the DNA molecule can further have a rare-cutting endonuclease target site 5′ of the first and second terminators (i.e., between the first and second terminators relative to the 5′ ends). The polynucleotide can further include a left and right homology arm flanking the first and second terminators.

This document also features a method of integrating a heterologous polynucleotide into an endogenous gene in the genome of a cell, where the method includes administering to a cell two recombinant nucleic acids. The first nucleic acid can include a sequence with, from 5′ to 3′, a first terminator, an shRNA silencing cassette, and a second terminator in reverse complement direction. The second nucleic acid can encode a rare-cutting endonuclease targeted to a site within an endogenous gene in the genome of the cell. Following delivery of both nucleic acids, the first nucleic acid can be integrated into the endogenous gene to provide a modified endogenous gene in which the first terminator or the second terminator is operatively linked to a promoter of the endogenous gene, and wherein the modified endogenous gene produces an mRNA transcript that is truncated relative to an mRNA transcript produced by the endogenous gene. The shRNA silencing cassette can reduce the expression of mRNA from an unmodified allele of the endogenous gene. The shRNA silencing cassette can be targeted to a sequence within the mRNA produced by the endogenous gene that is downstream of the corresponding DNA site targeted by the rare-cutting endonuclease. The method can include where the first nucleic acid is a linear double-stranded or a linear single-stranded DNA molecule. The method can further where the first nucleic acid is a circular double-stranded DNA molecule. The method can include where the first nucleic acid is a viral vector. The viral vector can be an adeno-associated virus vector, an adenovirus vector, or a lentivirus vector. The rare-cutting endonuclease can be used to facilitate integration of the first nucleic acid into the endogenous gene. The rare-cutting endonuclease can be a zinc-finger nuclease or a CRISPR nuclease. The method can include using a first nucleic acid that does not comprise a coding sequence and a coding sequence reverse complement operably linked to the first and second terminators. When the first recombinant nucleic acid is a circular double-stranded DNA molecule, the DNA molecule can further have a rare-cutting endonuclease target site 5′ of the first and second terminators (i.e., between the first and second terminators relative to the 5′ ends). The rare-cutting endonuclease target site within the circular DNA molecule can be the same target site as within the endogenous gene. The method can include integrating the first nucleic acid into the DMPK, ATXN8, ATXN8OS, or JPH3 gene. The method can include integrating the nucleic acid into the 3′ untranslated region of the DMPK gene. The method can include integrating the nucleic acid into the 3′ untranslated region of the DMPK gene downstream of the stop codon and upstream of the CTG repeat sequence. The shRNA silencing cassette can be targeted to an mRNA sequence of DMPK downstream of the CTG repeat sequence. The method can further include using first nucleic acids with a left and right homology arm flanking the first and second terminators.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety for all purposes. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DESCRIPTION OF DRAWINGS

FIG. 1 is an illustration showing the target sites for integrating polynucleotides into the DMPK gene. Target site 1 is within the 3′ UTR of the DMPK gene. Target site 2 is within intron 14 of the DMPK gene.

FIG. 2 is an illustration of the polynucleotides for the targeted insertion of two terminators in bidirectional orientation within the 3′ UTR of the DMPK gene. T1, terminator 1; T2, terminator 2; AS1, additional sequence 1; AS2, additional sequence 2; Nuclease, rare-cutting endonuclease; SaCas9, Streptococcus aureus Cas9; gRNA, guide RNA; RNAi, silencing cassette capable of decreasing the expression of a target gene.

FIG. 3 is an illustration of the polynucleotides for the targeted insertion of two terminators in bidirectional orientation into an intron within the DMPK gene. T1, terminator 1; T2, terminator 2; AS1, additional sequence 1; AS2, additional sequence 2; Nuclease, rare-cutting endonuclease; SaCas9, Streptococcus aureus Cas9; gRNA, guide RNA; RNAi, silencing cassette capable of decreasing the expression of a target gene; CDS1, coding sequence 1; SA1, splice acceptor 1; CDS2, coding sequence 2; SA2, splice acceptor 2.

FIG. 4 is an illustration of an AAV vector comprising a first and second terminator in bidirectional orientation for targeted integration into the DMPK 3′ untranslated region. The AAV vector can comprise homology arms to facilitate integration by HR or NHEJ.

FIG. 5 is an illustration of an AAV vector comprising a first and second terminator in bidirectional orientation and an RNAi silencing cassette for targeted integration into the DMPK 3′ untranslated region. The RNAi silencing cassette can comprise any suitable method that decreases expression of the DMPK gene. The target for the RNAi cassette can be the 3′ end of the 3′ untranslated region. The outcome of integration into either the wild type or mutant allele can result in a normal DMPK protein being produced by the first allele, and silencing of the second allele. ASO, antisense oligonucleotide.

FIG. 6 is an illustration of an AAV vector comprising a first and second terminator in bidirectional orientation operably linked to a first and second splice acceptor and first and second coding sequence encoding the peptide produced by exon 15 of a wild type DMPK gene. The AAV vector can comprise homology arms to facilitate integration by HR or NHEJ.

FIG. 7 is an illustration of an AAV vector comprising a first and second terminator in bidirectional orientation operably linked to a first and second splice acceptor and first and second coding sequence encoding the peptide produced by exon 15 of a wild type DMPK gene, and an RNAi silencing cassette for targeted integration into the DMPK 3′ untranslated region. The RNAi silencing cassette can comprise any suitable method that decreases expression of the DMPK gene. The target for the RNAi cassette can be the 3′ end of the 3′ untranslated region. The outcome of integration into either the wild type or mutant allele can result in a normal DMPK protein being produced by the first allele, and silencing of the second allele. ASO, antisense oligonucleotide.

FIG. 8 is an illustration of a linear DNA molecule comprising two terminators in a bidirectional, tail-to-tail orientation. Also shown is an AAV vector comprising two terminators in a bidirectional, tail-to-tail orientation, with a spacer comprising sequence encoding Cas9 and a gRNA. The target for integration of both constructs is the 3′ untranslated region of the DMPK gene.

FIG. 9 is an illustration of the gRNAs targeting DMPK for the targeted integration of polynucleotides comprising two terminators in a bidirectional, tail-to-tail orientation.

FIG. 10 is an illustration of a circular polynucleotide comprising two terminators in opposite orientations. A nuclease target site is present within a spacer sequence in the region between the two terminators within the head-to-head orientation. Also shown is the cleavage of the circular DNA to produce a linear polynucleotide with two terminators in tail-to-tail orientation for integration into the DMPK 3′ untranslated region.

FIG. 11 is an illustration of a circular polynucleotide comprising two terminators in opposite orientations operably linked to splice acceptors and coding sequences. A nuclease target site is present within a spacer sequence in the region between the two splice acceptors and within the head-to-head orientation. Also shown is the cleavage of the circular DNA to produce a linear polynucleotide for integration into intron 14 of the DMPK gene.

### DETAILED DESCRIPTION

Disclosed herein are methods and compositions for modifying the 3′ untranslated region or coding sequence of endogenous genes. In some embodiments, the methods include inserting a polynucleotide into an endogenous gene, wherein the polynucleotide harbors two terminators, and integration of the polynucleotide in either direction by the non-homologous end joining pathway can result in early termination of the endogenous gene's transcript. In other embodiments, the methods include delivering a polynucleotide, wherein the circular polynucleotide harbors two splice acceptors, two coding sequences and two terminators, and integration of the polynucleotide in either direction by the non-homologous end joining pathway can result in modification of the 3′ end of the endogenous gene. The methods described herein can be used together with viral or non-viral delivery methods.

In one embodiment, this document features a method of integrating a polynucleotide into an endogenous gene, the method including administering a polynucleotide, wherein the polynucleotide is circular and comprises a first and second splice acceptor sequence, a first and second partial coding sequence, and one bidirectional terminator or a first and second terminator, and administering one or more rare-cutting endonuclease targeted to a site within the endogenous gene and polynucleotide, wherein the polynucleotide is integrated within the endogenous gene. The method can include designing the polynucleotide to have the first splice acceptor operably linked to the first partial coding sequence and the second splice acceptor operably linked to the second partial coding sequence. The arrangement can also include having the first partial coding sequence operably linked to the first terminator, and the second partial coding sequence operably linked to the second terminator.

In another embodiment, this document features a linear polynucleotide, harbored on a viral vector or a polynucleotide. The linear polynucleotide can comprise a first and second terminator in a tail-to-tail orientation. The method can further include administering at least one rare-cutting endonuclease targeted to a site within the endogenous gene, wherein the polynucleotide is integrated within the endogenous gene. The method can include administering a polynucleotide in the format of a linear double-stranded or single-stranded DNA molecule. The method can include administering the polynucleotide within an AAV vector. In one embodiment, the terminators can be positioned within the polynucleotide such that the first terminator is the first functional element adjacent to the first ITR or first exposed double-stranded or single-stranded DNA end, and the second terminator is the second functional element adjacent to the second ITR or second exposed double-stranded or single stranded DNA end. In another embodiment, homology arms are added to the AAV vectors adjacent to the first and second ITRs, and flanking the first and second terminators. In other embodiments, homology arms are added to the linear double-stranded DNA or single-stranded DNA molecules adjacent to the exposed DNA ends.

In such cases, the first terminator can be the first functional element adjacent to the first homology arm and the second terminator can be the first functional element adjacent to the second homology arm. In one embodiment, there can be no spacer sequence between the first and second terminators. In other embodiments, there can be a spacer between the first and second terminators. The spacer sequence can be a coding sequence for one or more rare-cutting endonucleases. In another embodiment, the spacer sequence can be a silencing cassette. If the spacer sequence comprises sequence encoding a nuclease, the nuclease can be a CRISPR nuclease. The spacer sequence can encode either a Cas enzyme, a corresponding gRNA, or both the Cas enzyme and corresponding gRNA. The nuclease can be a CRISPR/Cas12a nuclease, a CRISPR/Cas9 nuclease, or a zinc-finger nuclease. The endogenous gene can be selected from DMPK, ATXN8, ATXN8OS, or JPH3. The target for integration of the polynucleotide can be the 3′ untranslated region of the endogenous gene. The polynucleotide can comprise a first and second terminator, wherein the first and second terminators are not operably linked to a coding sequence.

In another embodiment, this document provides methods for modifying the DM1 gene (DMPK). The methods include administering a linear polynucleotide, wherein the polynucleotide comprises a first and second terminator in a tail-to-tail orientation, administering at least one rare-cutting endonuclease targeted to a site within the endogenous gene, wherein the polynucleotide is integrated within the 3′ UTR of DMPK. In some embodiments, the linear polynucleotide can be administered within an AAV vector. In another embodiment, the polynucleotide can comprise a first splice acceptor operably linked to a first coding sequence operably linked to the first terminator and a second splice acceptor operably linked to a second coding sequence operably linked the second terminator. The polynucleotide can be integrated into an intron of the DMPK gene. In another embodiment, the polynucleotide can be circular and comprise two terminators in opposite directions and a rare-cutting endonuclease target site between the 5′ ends of the two terminators. Upon linearization by cleavage by the rare-cutting endonuclease, the polynucleotide can integrate into the 3′ UTR of DMPK. In another embodiment, the circular polynucleotide can comprise a first splice acceptor operably linked to a first coding sequence operably linked to the first terminator and a second splice acceptor operably linked to a second coding sequence operably linked to the second terminator, wherein the first and second coding sequences are oriented in opposite directions. A rare-cutting endonuclease target site can be placed between the splice acceptors. Upon linearization by cleavage by the rare-cutting endonuclease, the polynucleotide can integrate into an intron within the DMPK gene.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999. As used herein, the terms “nucleic acid” and “polynucleotide,” can be used interchangeably. Nucleic acid and polynucleotide can refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” can be used interchangeably to refer to amino acid residues covalently linked together. The term also applies to proteins in which one or more amino acids are chemical analogues or modified derivatives of corresponding naturally-occurring amino acids.

The terms “operatively linked” or “operably linked” are used interchangeably and refer to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

As used herein, the term “cleavage” refers to the breakage of the covalent backbone of a nucleic acid molecule. Cleavage can be initiated by a variety of methods including, but not limited to, enzymatic or chemical hydrolysis of a phosphodiester bond. Cleavage can refer to both a single-stranded nick and a double-stranded break. A double-stranded break can occur as a result of two distinct single-stranded nicks. Nucleic acid cleavage can result in the production of either blunt ends or staggered ends. In some embodiments, rare-cutting endonucleases are used for targeted double-stranded or single-stranded DNA cleavage.

The term “first functional element” refers to the position of a sequence of DNA on a polynucleotide. For illustrative purposes, with the sentence “the first terminator is the first functional element adjacent to the first exposed double-stranded DNA end”, the “first functional element” refers to the first terminator. The first terminator is the first functional element adjacent to the exposed double-stranded DNA end, meaning there is no other functional element between the double-stranded DNA end and the first terminator. As herein defined, there are no functional elements between the first terminator and double-stranded DNA end, including no splice acceptor, no promoter, no functional coding sequence, or no transcriptional regulatory sequence. There may be a spacer sequence between the first terminator and double-stranded DNA end. The spacer sequence may comprise sequence that encodes a barcode for purposes of distinguishing between integration events. The spacer sequence may comprise a partial or full 3′ UTR sequence.

An “exogenous” molecule can refer to a small molecule (e.g., sugars, lipids, amino acids, fatty acids, phenolic compounds, alkaloids), or a macromolecule (e.g., protein, nucleic acid, carbohydrate, lipid, glycoprotein, lipoprotein, polysaccharide), or any modified derivative of the above molecules, or any complex comprising one or more of the above molecules, generated or present outside of a cell, or not normally present in a cell. Exogenous molecules can be introduced into cells. Methods for the introduction or “administering” of exogenous molecules into cells can include lipid-mediated transfer, electroporation, direct injection, cell fusion, particle bombardment, calcium phosphate co-precipitation, DEAE-dextran-mediated transfer and viral vector-mediated transfer. As defined herein, “administering” can refer to the delivery, the providing, or the introduction of exogenous molecules into a cell. If a polynucleotide or a rare-cutting endonuclease is administered to a cell, then the polynucleotide or rare-cutting endonuclease is delivered to, provided, or introduced into the cell. The rare-cutting endonuclease can be administered as purified protein, nucleic acid, or a mixture of purified protein and nucleic acid. The nucleic acid (i.e., RNA or DNA), can encode for the rare-cutting endonuclease, or a part of a rare-cutting endonuclease (e.g., a gRNA). The administering can be achieved though methods such as lipid-mediated transfer, electroporation, direct injection, cell fusion, particle bombardment, calcium phosphate co-precipitation, DEAE-dextran-mediated transfer, viral vector-mediated transfer, or any means suitable of delivering purified protein or nucleic acids, or a mixture of purified protein and nucleic acids, to a cell.

An “endogenous” molecule is a molecule that is present in a particular cell at a particular developmental stage under particular environmental conditions. An endogenous molecule can be a nucleic acid, a chromosome, the genome of a mitochondrion, chloroplast or other organelle, or a naturally-occurring episomal nucleic acid. Additional endogenous molecules can include proteins, for example, transcription factors and enzymes.

As used herein, a “gene,” refers to a DNA region that encodes a gene product, including all DNA regions which regulate the production of the gene product.

Accordingly, a gene includes, but is not necessarily limited to, coding sequences, intron sequences, exon sequences, promoter sequences, terminators, translational regulatory sequences such as ribosome binding sites and internal ribosome entry sites, enhancers, silencers, insulators, boundary elements, replication origins, matrix attachment sites and locus control regions.

An “endogenous gene” refers to a gene that is normally present in the genome of a particular cell.

“Gene expression” refers to the conversion of the information, contained in a gene, into a gene product. A gene product can be the direct transcriptional product of a gene. For example, the gene product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Encoding” refers to the conversion of the information contained in a nucleic acid, into a product, wherein the product can result from the direct transcriptional product of a nucleic acid sequence. For example, the product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A “target site” or “target sequence” is a nucleic acid sequence to which a binding molecule will bind, provided sufficient conditions for binding exist, such as an endonuclease, including for example a rare-cutting endonuclease. The target site can be an endogenous gene which may be native to the cell or heterologous.

As used herein, the term “recombination” refers to a process of exchange of genetic information between two polynucleotides. The term “homologous recombination (HR)” refers to a specialized form of recombination that can take place, for example, during the repair of double-strand breaks. Homologous recombination requires nucleotide sequence homology present on a “donor” molecule or a polynucleotide. The donor molecule or polynucleotide can be used by the cell as a template for repair of a double-strand break. Information within the donor molecule that differs from the genomic sequence at or near the double-strand break can be stably incorporated into the cell's genomic DNA.

The term “integrating” as used herein refers to the process of adding DNA to a target region of DNA. As described herein, integration can be facilitated by several different means, including non-homologous end joining, microhomology-mediated end joining, or homologous recombination. By way of example, integration of a user-supplied DNA molecule into a target gene can be facilitated by non-homologous end joining. Here, a targeted-double strand break is made within the target gene and a user-supplied DNA molecule is administered. The user-supplied DNA molecule can comprise exposed DNA ends to facilitate capture during repair of the target gene by non-homologous end joining. The exposed ends can be present on the DNA molecule upon administration (i.e., administration of a linear DNA molecule) or created upon administration to the cell (i.e., a rare-cutting endonuclease cleaves the user-supplied DNA molecule within the cell to expose the ends). Additionally, the user-supplied DNA molecule can be harbored on a viral vector, including an adeno-associated virus vector. The adeno-associated virus vector can integrate within a targeted-double strand break. In another example, integration occurs though homologous recombination. Here, the user-supplied DNA can harbor a left and right homology arm.

As used herein, the term “pathogenic” refers to anything that can cause disease. A pathogenic mutation can refer to a modification in a gene which causes disease. A pathogenic gene refers to a gene comprising a modification which causes disease. By means of example, a pathogenic DMPK gene in patients with myotonic dystrophy type 1 refers to an DMPK gene with an allele having an expanded CTG trinucleotide repeat, wherein the expanded CTG trinucleotide repeat causes the disease.

As used herein, the term “tail-to-tail” refers to an orientation of two units in opposite and reverse directions. The two units can be two sequences on a single nucleic acid molecule, where the 3′ end of each sequence are placed adjacent to each other, either directly linked with no spacer, or having a spacer sequence separating the 3′ ends. For example, a first sequence having the elements, in a 5′ to 3′ direction, [splice acceptor 1]-[coding sequence 1]-[terminator 1] and a second sequence having the elements [splice acceptor 2]-[coding sequence 2]-[terminator 2] can be placed in tail-to-tail orientation resulting in [splice acceptor 1]-[coding sequence 1][terminator 1]-[terminator 2 RC]-[coding sequence 2 RC]-[splice acceptor 2 RC], where RC refers to reverse complement.

The term “intron-exon junction” refers to a specific location within a gene. The specific location is between the last nucleotide in an intron and the first nucleotide of the following exon. When integrating a polynucleotide described herein, the polynucleotide can be integrated within the “intron-exon junction.” If the polynucleotide comprises cargo, the cargo will be integrated immediately following the last nucleotide in the intron. In some cases, integrating a polynucleotide within the intron-exon junction can result in removal of sequence within the exon (e.g., integration via HR and replacement of sequence within the exon with the cargo within the polynucleotide).

The term “homologous” as used herein refers to a sequence of nucleic acids or amino acids having similarity to a second sequence of nucleic acids or amino acids. In some embodiments, the homologous sequences can have at least 80% sequence identity (e.g., 81%, 85%, 90%, 95%, 96%, 97%, 98%, or 99% sequence identity) to one another.

The term “partial coding sequence” as used herein refers to a sequence of nucleic acids that encodes a partial protein. The partial coding sequence can encode a protein that comprises one or less amino acids as compared to the wild type protein or functional protein. The partial coding sequence can encode a partial protein with homology to the wild type protein or functional protein. The term “partial coding sequence” when referring to DMPK refers to a sequence of nucleic acids that encodes a partial DMPK protein. The partial DMPK protein has one or less amino acids compared to a wild type DMPK protein. If modifying the 3′ end of the gene, the one or less amino acids can be from the N-terminus end of the protein. If the DMPK gene has 15 exons, then the partial coding sequence can include nucleotides encoding the peptide produced by exons 2-15, or 3-15 or 4-15, or 5-15, or 6-15, or 7-15, or 8-15, or 9-15, or 10-15, or 11-15, or 12-15, or 13-15, or 14-15, or 15.

The phrase “wherein the recombinant nucleic acid does not comprise a coding sequence and a coding sequence reverse complement operably linked to the first and second terminators” refers to the composition of a nucleic acid for insertion into an endogenous gene, wherein the nucleic acid does not provide coding sequence for amino acids that would be incorporated into the resulting polypeptide after integration. By way of example, a recombinant nucleic acid that does not comprise a coding sequence and a coding sequence reverse complement operably linked to a first and second terminators can be a linear nucleic acid with a 5′ and 3′ exposed DNA end. The sequence within the linear nucleic acid at the 5′ end can be a terminator which is not operably linked to a coding sequence. The sequence within the linear polynucleotide at the 3′ end can be a terminator in reverse complement which is not operably linked to a coding sequence. The linear nucleic acid can be integrated downstream of an endogenous gene's stop codon within the 3′ UTR, which results in no amino acids from the nucleic acid being incorporated into the expressed protein product. By way of another example, a recombinant nucleic acid that does not comprise a coding sequence and a coding sequence reverse complement operably linked to a first and second terminators can be a linear AAV nucleic acid with two inverted terminal repeat sequences at the 5′ and 3′ end. The AAV nucleic acid can comprise a first terminator downstream of the inverted terminal repeat sequence, and a second terminator in reverse complement upstream of the second inverted terminal repeat. The AAV polynucleotide can be directly integrated into the 3′ UTR of an endogenous gene, wherein the AAV polynucleotide or inverted terminal repeats do not provide coding sequence for amino acids that would be incorporated into the resulting polypeptide after integration.

The term “silencing agent” refers to a nucleic acid that reduces the levels of RNA or protein from an endogenous gene. The silencing agent can be in the format of RNA or DNA or a combination of RNA and DNA. The silencing agent may be shRNA, siRNA, miRNA or an antisense oligonucleotide. The silencing agent can be delivered to cells as pure RNA or DNA, or the silencing agent can be delivered on a vector which produces the silencing agent.

The terms “silencing” and “silence” and “reduced expression” and “inhibit the expression of”, in as far as they refer to silencing agent described herein refers to the at least partial suppression of the expression of an endogenous gene, as manifested by a reduction of the amount of mRNA or protein produced from the endogenous gene, as compared the amount of mRNA or protein produced by the endogenous gene in cells that have not been treated with the silencing agent. In some embodiments, the silencing agent can be a miRNA. miRNAs are a group of small non-coding RNA molecules produced endogenously miRNAs are encoded in the genome and are transcribed by RNA polymerase II (pol II) as long precursor transcripts, which are known as primary miRNAs (pri-miRNAs) of several kilobases in length. A short hairpin RNA is an artificial RNA molecule with a hairpin turn that can be used to silence target gene expression. Expression of shRNA in cells can be accomplished by delivery of plasmids, RNA, or through viral or bacterial vectors. The shRNA can be processed into siRNAs which facilitate silencing of the target mRNA transcript. shRNA can be considered a precursor to siRNA. Whereas siRNA can be delivered directly to cells, it can also be produced by delivering shRNA, which is then processed into siRNA. Antisense oligonucleotides (ASOs) are short, synthetic, single-stranded oligodeoxynucleotides that can alter RNA and reduce expression. Therapeutic ASOs are synthetic single-stranded deoxyribonucleotide analogs, usually 15-30 bp in length. Their sequence (3′ to 5′) is antisense and complementary to the sense sequence of the target nucleotide sequence.

In some embodiments, the cells described herein comprising a gene editing event within an endogenous gene can be delivered an RNA interference agent targeting mRNA produced by the endogenous gene. RNA interference (RNAi) refers to the process of sequence-specific post transcriptional gene silencing mediated by small interfering RNAs (siRNA), which can be produced from a precursor shRNA. Long double stranded RNA (dsRNA) in cells stimulates the activity of a ribonuclease III enzyme referred to as dicer. Dicer is involved in the processing of the long dsRNA into short pieces of siRNA. siRNAs derived from dicer activity are typically about 21-23 nucleotides in length and include duplexes of about 19 base pairs. The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex. siRNA mediated RNAi has been studied in a variety of systems. RNAi technology has been used in mammalian cell culture, where a siRNA-mediated reduction in gene expression has been accomplished by transfecting cells with synthetic RNA oligonucleotides. The ability to use siRNA-mediated gene silencing in mammalian cells combined with the high degree of sequence specificity allows RNAi technology to be used to selectively silence expression of mutant alleles or toxic gene products in dominantly inherited diseases, including neurodegenerative diseases. Several neurodegenerative diseases, such as myotonic dystrophy, Parkinson's disease, Alzheimer's disease, Huntington's disease, Spinocerebellar Ataxia Type 1, Type 2, and Type 3, and dentatorubral pallidoluysian atrophy (DRLPA), have proteins identified that are involved in the overall pathogenic progression of the disease. siRNA-mediated gene silencing of mutant forms of human ataxin-3, Tau and TorsirL4, genes which cause neurodegenerative diseases such as spinocerebellar ataxia type 3, frontotemporal dementia and DYTI dystonia respectively, has been demonstrated in cultured cells.

In an embodiment, the silencing agent can be an shRNA or siRNA (the processed product of shRNA). siRNAs may be constructed in vitro using synthetic oligonucleotides or appropriate transcription enzymes or in vivo using appropriate transcription enzymes or expression vectors. The siRNAs include a sense RNA strand and a complementary antisense RNA strand annealed together by standard Watson-Crick base-pairing interactions to form the base pairs. The sense and antisense strands of the present siRNA may be complementary single stranded RNA molecules to form a double stranded (ds) siRNA or a DNA polynucleotide encoding two complementary portions that may include a hairpin structure linking the complementary base pairs to form the siRNA. Preferably, the duplex regions of the siRNA formed by the ds RNA or by the DNA polypeptide include about 15-30 base pairs, more preferably, about 19-25 base pairs. The siRNA duplex region length may be any positive integer between 15 and 30 nucleotides. The siRNA of the invention derived from ds RNA may include partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the invention may include a 3′ overhang. As used herein, a “3′ overhang” refers to at least one unpaired nucleotide extending from the 3′-end of an RNA strand. In an embodiment, the siRNA may include at least one 3′ overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length. Both strands of the siRNA molecule may include a 3′ overhang, the length of the overhangs can be the same or different for each strand. The 3′ overhang may be present on both strands of the siRNA, and is 2 nucleotides in length. The 3′ overhangs may also be stabilized against degradation. For example, the overhangs may be stabilized by including purine nucleotides, such as adenosine or guanosine nucleotides, by substitution of pyrimidine nucleotides by modified analogues, e.g., substitution of uridine nucleotides in the 3′ overhangs with 2′-deoxythymidine, is tolerated and does not affect the efficiency of RNAi degradation, hi particular, the absence of a 2′ hydroxyl in the 2′-deoxythymidine significantly enhances the nuclease resistance of the 3′ overhang in tissue culture medium.

In some embodiments, the RNA duplex portion of the siRNA may be part of a hairpin structure. The hairpin structure may further contain a loop portion positioned between the two sequences that form the duplex. The loop can vary in length. In some embodiments, the loop may be 5, 6, 7, 8, 9, 10, 11, 12 or 13 nucleotides in length. The hairpin structure may also contain 3′ or 5′ overhang portions. In some embodiments, the overhang is a 3′ or a 5′ overhang 0, 1, 2, 3, 4 or 5 nucleotides in length.

In some embodiments, the siRNA of the present invention may also be expressed from a recombinant plasmid either as two separate, complementary RNA molecules, or as a single RNA molecule with two complementary regions. Selection of vectors suitable for expressing siRNA of the invention, methods for inserting nucleic acid sequences for expressing the siRNA into the plasmid, and methods of delivering the recombinant plasmid to the cells of interest are within the skill in the art. The siRNA of the present invention may be a polynucleotide sequence cloned into a plasmid vector and expressed using any suitable promoter. Suitable promoters for expressing siRNA of the invention from a plasmid include, but are not limited to, the Hl and U6 RNA pol III promoter sequences and viral promoters including the viral LTR, adenovirus, SV40, and CMV promoters. Additional promoters known to one of skill in the art may also be used, including tissue specific, inducible or regulatable promoters for expression of the siRNA in a particular tissue or in a particular intracellular environment. The vector may also include additional regulatory or structural elements, including, but not limited to introns, enhancers, and polyadenylation sequences. These elements may be included in the DNA as desired to obtain optimal performance of the siRNA in the cell and may or may not be necessary for the function of the DNA. Optionally, a selectable marker gene or a reporter gene may be included either with the siRNA encoding polynucleotide or as a separate plasmid for delivery to the target cells. Additional elements known to one of skill in the art may also be included.

The siRNA may also be expressed from a polynucleotide sequence cloned into a viral vector that may include the elements described above. Suitable viral vectors for gene delivery to a cell include, but are not limited to, replication-deficient viruses that are capable of directing synthesis of all virion proteins, but are incapable of making infections particles. Exemplary viruses include, but are not limited to lentiviruses, adenoviruses, adeno-associated viruses, retroviruses, and alphaviruses.

In some embodiments, shRNA may also be expressed from a polynucleotide sequence cloned into a viral vector that may include the elements described above. Suitable viral vectors for gene delivery to a cell include, but are not limited to, replication-deficient viruses that are capable of directing synthesis of all virion proteins, but are incapable of making infections particles. Exemplary viruses include, but are not limited to lentiviruses, adenoviruses, adeno-associated viruses, retroviruses, and alphaviruses.

The siRNA may also be delivered to cells in vitro or in vivo using lipid nanoparticles. When using lipid nanoparticles, the siRNA may be in the form of purified RNA. Physical methods to introduce a preselected DNA or RNA duplex into a host cell further include, but are not limited to, calcium phosphate precipitation, lipofection, DEAE-dextran, particle bombardment, microinjection, electroporation, immunoliposomes, lipids, cationic lipids, phospholipids, or liposomes and the like. One skilled in the art will understand that any method may be used to deliver the DNA or RNA duplex into the cell. One mode of administration to the CNS uses a convection-enhanced delivery (CED) system. This method includes: a) creating a pressure gradient during interstitial infusion into white matter to generate increased flow through the brain interstitium (convection-supplementing simple diffusion); b) maintaining the pressure gradient over a lengthy period of time (24 hours to 48 hours) to allow radial penetration of the migrating compounds (such as: neurotrophic factors, antibodies, growth factors, genetic vectors, enzymes, etc.) into the gray matter; and c) increasing drug concentrations by orders of magnitude over systemic levels. Using a CED system, DNA, RNA duplexes or viruses can be delivered to many cells over large areas of the brain. Any CED device may be appropriate for delivery of DNA, RNA or viruses. In some embodiments, the device is an osmotic pump or an infusion pump. Both osmotic and infusion pumps are commercially available from a variety of suppliers, for example Alzet Corporation, Hamilton Corporation, Alza, Inc., Palo Alto, Calif.). Biological methods to introduce the nucleotide of interest into a host cell include the use of DNA and RNA viral vectors. For mammalian gene therapy, it is desirable to use an efficient means of inserting a copy gene into the host genome. Viral vectors have become the most widely used method for inserting genes into mammalian, e.g., human cells. Delivery of the recombinant nucleotides to the host cell may be confirmed by a variety of assays known to one of skill in the art. Assays include Southern and Northern blotting, RT-PCR, PCR, ELISA, and Western blotting, by way of example. The methods and compositions described in this document can use polynucleotides having a cargo sequence. The term “cargo” can refer to elements such as the complete or partial coding sequence of a gene, a partial sequence of a gene harboring single-nucleotide polymorphisms relative to the WT or altered target, a splice acceptor, a terminator, a transcriptional regulatory element, purification tags (e.g., glutathione-S-transferase, poly(His), maltose binding protein, Strep-tag, Myc-tag, AviTag, HA-tag, or chitin binding protein) or reporter genes (e.g., GFP, RFP, lacZ, cat, luciferase, puro, neomycin). As defined herein, “cargo” can refer to the sequence within a polynucleotide that is integrated at a target site. For example, “cargo” can refer to the sequence on a polynucleotide between two homology arms, two rare-cutting endonuclease target sites, two exposed DNA ends, or two inverted terminal repeats.

The term “homology arm” or “homology arms” refers to a sequence of nucleic acids that comprises homology to a second nucleic acid. Homology arms, for example, can be present on a donor molecule. Homology arms can facilitate homologous recombination with the second nucleic acid. In an embodiment, homology arms can have homology to an endogenous gene.

The term “bidirectional terminator” refers to a single terminator that can terminate RNA polymerase transcription in either the sense or antisense direction. In contrast to two unidirectional terminators in tail-to-tail orientation, a bidirectional terminator can comprise a non-chimeric sequence of DNA. Examples of bidirectional terminators include the ARO4, TRP1, TRP4, ADH1, CYC1, GAL1, GAL7, and GAL10 terminator.

A 5′ or 3′ end of a nucleic acid molecule references the directionality and chemical orientation of the nucleic acid. As defined herein, the “5′ end of a gene” can comprise the exon with the start codon, but not the exon with the stop codon. As defined herein, the “3′ end of a gene” can comprise the exon with the stop codon, but not the exon with the start codon.

The term “RNAi” refers to RNA interference, a process that uses RNA molecules to inhibit or reduce gene expression or translation. RNAi can be induced with the use of small interfering RNAs (siRNA) or short hairpin RNAs (shRNA).

The term “DMPK” gene refers to a gene that encodes the enzyme DM1 protein kinase A representative sequence of the DMPK gene can be found with NCBI Reference Sequence: NG_009784. Specifically, exon 1 includes the sequence from 1 to 298. Exon 2 includes the sequence from 2622 to 2713. Exon 3 includes the sequence from 2969 to 3052. Exon 4 includes the sequence from 3132 to 3227. Exon 5 includes the sequence from 3850 to 3998. Exon 6 includes the sequence from 4271 to 4364. Exon 7 includes the sequence from 4618 to 4824. Exon 8 includes the sequence from 4901 to 5164. Exon 9 includes the sequence from 7457 to 7542. Exon 10 includes the sequence from 9739 to 9850. Exon 11 includes the sequence from 10563 to 10720. Exon 12 includes the sequence from 10826 to 10923. Exon 13 includes the sequence from 11095 to 11141. Exon 14 includes the sequence from 11431 to 11520. Exon 15 includes the sequence from 11851 to 12774. Intron 1 includes the sequence from 299 to 2621. Intron 2 includes the sequence from 2714 to 2968. Intron 3 includes the sequence from 3053 to 3131. Intron 4 includes the sequence from 3228 to 3849. Intron 5 includes the sequence from 3999 to 4270. Intron 6 includes the sequence from 4365 to 4617. Intron 7 includes the sequence from 4825 to 4900. Intron 8 includes the sequence from 5165 to 7456. Intron 9 includes the sequence from 7543 to 9738. Intron 10 includes the sequence from 9851 to 10562. Intron 11 includes the sequence from 10721 to 10825. Intron 12 includes the sequence from 10924 to 11094. Intron 13 includes the sequence from 11142 to 11430. Intron 14 includes the sequence from 11521 to 11850.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q -1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. The percent sequence identity value is rounded to the nearest tenth.

As defined herein, “administering” can refer to the delivery, the providing, or the introduction of exogenous molecules into a cell. If a polynucleotide or a rare-cutting endonuclease is administered to a cell, then the polynucleotide or rare-cutting endonuclease is delivered to, provided to, or introduced into the cell. The rare-cutting endonuclease can be administered as purified protein, nucleic acid, or a mixture of purified protein and nucleic acid. The nucleic acid (i.e., RNA or DNA), can encode for the rare-cutting endonuclease, or a part of a rare-cutting endonuclease (e.g., a gRNA). The administering can be achieved though methods such as lipid-mediated transfer, electroporation, direct injection, cell fusion, particle bombardment, calcium phosphate co-precipitation, DEAE-dextran-mediated transfer, viral vector-mediated transfer, or any means suitable of delivering purified protein or nucleic acids, or a mixture of purified protein and nucleic acids, to a cell. Administer can refer to the delivery, the providing, or the introduction of exogenous molecules to an organism, which will then result in administering of exogenous molecules to cells within the organism.

In some embodiments, the methods provided herein result in reduced expression of an endogenous gene's mRNA or protein product. For example, integration of polynucleotides comprising two terminators within the 3′ UTR of the DMPK gene can result in reduced expression of DMPK transcripts with the CTG repeat expansion. Reduced expression is expressed as expression that is less than the expression that occurs in otherwise comparable untreated cells. For example, in certain instances, expression of the endogenous gene is reduced by at least about 20%, 25%, 35%, or 50% by administration of the silencing agents described herein, as compared to the expression of the endogenous gene in a cell not administered the polynucleotides. In some embodiments, expression of the endogenous gene is reduced by at least about 60%, 70%, or 80% by administration of the polynucleotides, as compared to the expression of the endogenous gene in a cell not administered the polynucleotides. In some embodiments, expression of the endogenous gene is reduced by at least about 85%, 90%, or 95% by administration of the polynucleotides, as compared to the expression of the endogenous gene in a cell not administered the polynucleotides. In some embodiments, mRNA comprising repeat expansion polynucleotides produced by the endogenous gene is reduced. Detecting reduced levels of mRNA or protein can be assayed with methods common in the art, including Northern blotting, Western blotting, reverse transcription polymerase chain reaction (RT-PCR), RNA seq, or DNA microarray.

In one embodiment, this document features methods for modifying the 3′ UTR or 3′ end of endogenous genes, where endogenous genes can have at least one intron between two exons. The intron can be any intron which is removed from precursor messenger RNA by normal messenger RNA processing machinery. The intron can be between 20 bp and >500 kb and comprise elements including a splice donor site, branch sequence, and acceptor site. The polynucleotides disclosed herein for the modification of the 3′ UTR or 3′ end of endogenous genes can comprise multiple functional elements, including one or more target sites for rare-cutting endonucleases, homology arms, splice acceptor sequences, coding sequences, and transcription terminators.

In one embodiment, the polynucleotide comprises one or more target sites for one or more rare-cutting endonucleases. The target sites can be a suitable sequence and length for cleavage by a rare-cutting endonuclease. The target site can be amenable to cleavage by CRISPR systems, TAL effector nucleases, zinc-finger nucleases or meganucleases, or a combination of CRISPR systems, TAL effector nucleases, zinc finger nucleases or meganucleases, or any other site-specific nuclease. The target sites can be positioned such that cleavage by the rare-cutting endonuclease results in liberation of a linear polynucleotide from a circular plasmid.

The rare-cutting endonuclease can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary rare-cutting endonuclease [e.g., Cas9 from S. pyogenes, US2014/0068797 Sequence ID No. 8 or Sapranauskas et al., Nucleic Acids Res, 39(21): 9275-9282 (2011)], and various other rare-cutting endonuclease. The rare-cutting endonuclease can comprise at least 70, 75, 80, 85, 90, 95, 97, 99, or 100% identity to a wild-type rare-cutting endonuclease (e.g., Cas9 from S. pyogenes, supra) over 10 contiguous amino acids. The rare-cutting endonuclease can comprise at most: 70, 75, 80, 85, 90, 95, 97, 99, or 100% identity to a wild-type rare-cutting endonuclease (e.g., Cas9 from S. pyogenes, supra) over 10 contiguous amino acids. The rare-cutting endonuclease can comprise at least: 70, 75, 80, 85, 90, 95, 97, 99, or 100% identity to a wild-type rare-cutting endonuclease (e.g., Cas9 from S. pyogenes, supra) over 10 contiguous amino acids in a HNH nuclease domain of the rare-cutting endonuclease. The rare-cutting endonuclease can comprise at most: 70, 75, 80, 85, 90, 95, 97, 99, or 100% identity to a wild-type rare-cutting endonuclease (e.g., Cas9 from S. pyogenes, supra) over 10 contiguous amino acids in a HNH nuclease domain of the rare-cutting endonuclease. The rare-cutting endonuclease can comprise at least: 70, 75, 80, 85, 90, 95, 97, 99, or 100% identity to a wild-type rare-cutting endonuclease (e.g., Cas9 from S. pyogenes, supra) over 10 contiguous amino acids in a RuvC nuclease domain of the rare-cutting endonuclease. The rare-cutting endonuclease can comprise at most: 70, 75, 80, 85, 90, 95, 97, 99, or 100% identity to a wild-type rare-cutting endonuclease (e.g., Cas9 from S. pyogenes, supra) over 10 contiguous amino acids in a RuvC nuclease domain of the rare-cutting endonuclease.

A modified form of the rare-cutting endonuclease can comprise a mutation such that it can induce a single-strand break (SSB) on a target nucleic acid (e.g., by cutting only one of the sugar-phosphate backbones of a double-strand target nucleic acid). In some aspects, the mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type rare-cutting endonuclease (e.g., Cas9 from S. pyogenes, supra). In some aspects, the mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854 and Asn856, are mutated to inactivate one or more of the plurality of nucleic acid-cleaving domains (e.g., nuclease domains). The residues to be mutated can correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. One skilled in the art will recognize that mutations other than alanine substitutions can be suitable.

Nickase variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HR versus NHEJ. HR can be used to introduce selected changes into target sites in the genome through the use of specific donor sequences that effectively mediate the desired changes.

The one or more rare-cutting endonucleases, e.g. DNA endonucleases, can comprise two nickases that together effect one double-strand break at a specific locus in the genome, or four nickases that together effect or cause two double-strand breaks at specific loci in the genome. Alternatively, one rare-cutting endonuclease, e.g. DNA endonuclease, can effect or cause one double-strand break at a specific locus in the genome.

Non-limiting examples of Cas9 orthologs from other bacterial strains include but are not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacilhts acidocaldarius subsp. acidocaldarius DSM 446; Allochromatium vinosum DSM 180; Ammonifex degensii KC4; Anabaena variabilis ATCC 29413: Arthrospira maxita C'S-328; Arthrospira platensis str Paraca: Arthrospira sp PCC 8005; Bacillus pseudomycoides DSM 12442; Bacillus selenitireducens MLS 10: Burkholderiales bacterium 1_1_47; Caldicelulosiruptor becscii DSM 6725; Candidatus Desulforudis auckxviator MP104C; Caldicellulosiruptor hydrothermahs 108, Clostridium phage c-st; Clostriduim botuliinum A3 str. Loch Maree; Clostridium botulinum Ba4 str 657, Clostridium difficile QC)-63q42; Crocosphaera watsonii WH 8501; Cyanothece sp. ATCC 51142; Cyanothece sp. CCY0110: Cyanothece sp PCC 7424; Cyanothece sp. PCC 7822; Exiguobacterium sibiricum 255-15; Finegoldia magna ATCC 29328: Ktedonobacter racemifer DSM 44963, Lactobacillus delbrueckii subsp. bulgaricus PB2003t044-T3-4; Lactobacillus salivarius ATCC 11741; Listeria innocua; Lyngbva sp. PCC 8106; Marinobacter sp. ELB17: Methanohalobium evestigatun Z-7303; Microcystis phage Ma-L4MMN01; Microcystis aeruginosa NIES-843; Microscilla marina ATCC 23134: Microcoleus chthonophistes PCC 7420; Neisseru meningitidis; Nitrosococcus halophihis Nc4: Nocardiopsis dassonvillei subsp. dassonvillei DSM 43111: Nodularia spunigena CCY9414; Nostoc sp. PCC 7120; Oscillatoria sp. PCC 6506; Pelotomaculum thermopropionicum_SI, Petrotoga mobilis SJ95, Polaromonas naphthalenivorans CJ2; Polarononas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021: Synechococcus sp. PCC 7335; and Thermosipho africanus TCF52B (Chylinski et al., RNA Biol., 2013; 10(5) 726-737)

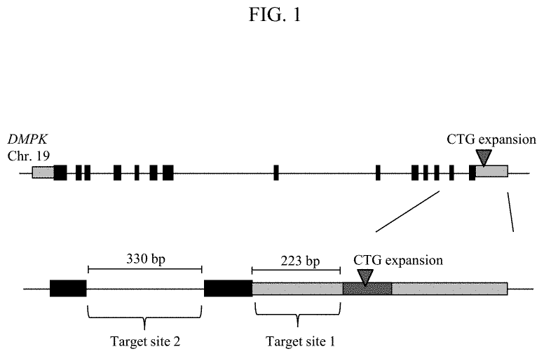

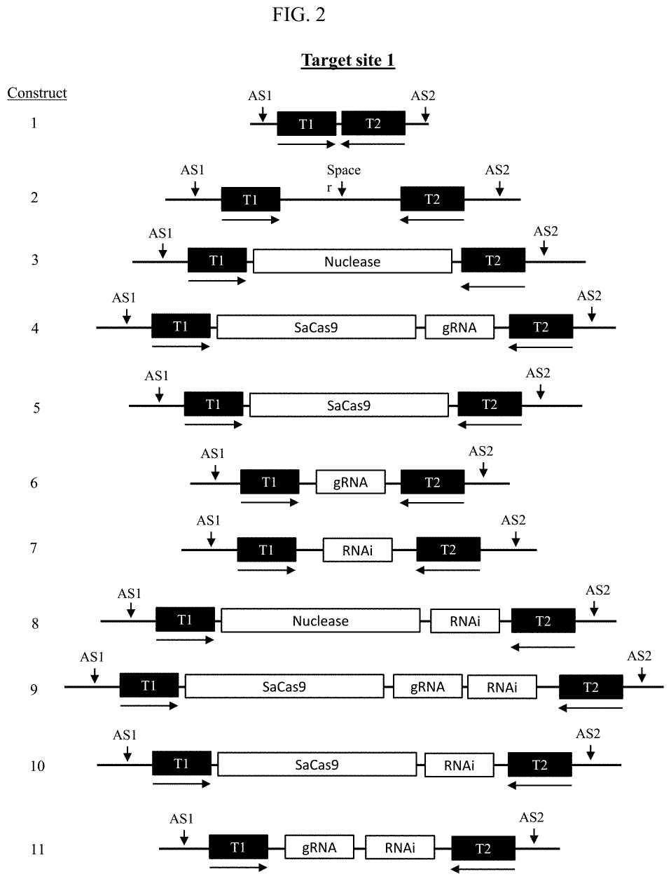

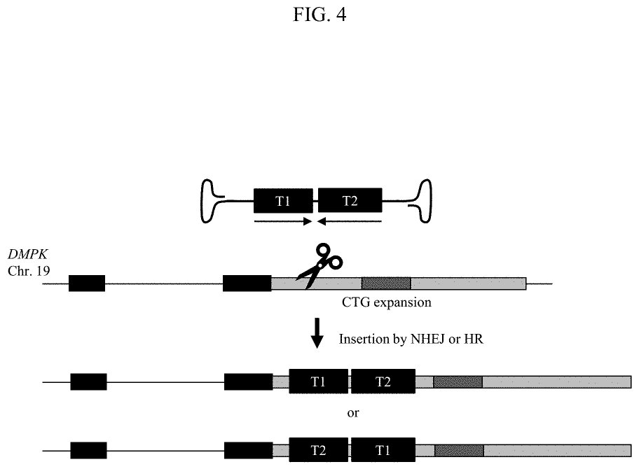

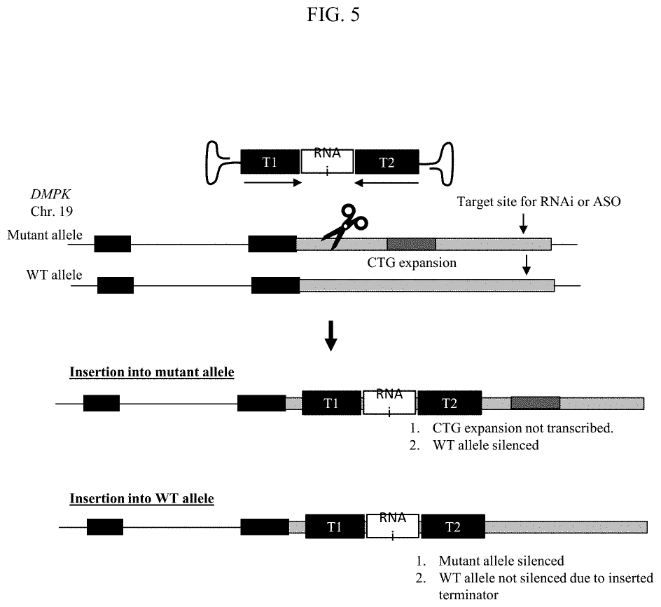

In one embodiment, the methods described herein provide linear polynucleotides for integration into the 3′ UTR of a target gene. The linear polynucleotide can comprise a first and second terminator (FIG. 2). The first and second terminator can be in opposite orientation of each other. The first and second terminators can be in tail-to-tail orientation of each other. The first and second terminator can have no spacer sequence between them, or they can have a spacer sequence between them. The terminators can be defined by having a 5′ and 3′ end. The spacer sequence can be between the 3′ ends. The spacer sequence can include functional sequences, including silencing cassettes, rare-cutting endonuclease coding sequences, Cas sequences, or gRNA sequences, or a combination of silencing cassettes, rare-cutting endonuclease coding sequences, Cas sequences, and gRNA sequences. The first and second terminators, along with any spacer sequence, can be harbored on a double-stranded or single stranded DNA molecule with exposed DNA ends. The first and second terminators can be the first functional element adjacent to the exposed ends. In other embodiments, the first and second terminators, along with any spacer sequence, can be harbored on a viral vector, including an AAV vector with a first and second inverted terminal repeat. Here, first and second terminators can be the first functional element adjacent to the ITRs. In another embodiment, the linear DNA molecules or AAV vectors can also comprise a left and right homology arm. The left and right homology arm can be adjacent to the exposed ends or ITRs, followed by the first and second terminators. The polynucleotides described herein can be used to modify the 3′ UTR of the DMPK gene (FIG. 1 and FIG. 4). Further, the RNAi comprised within the spacer sequence can target a sequence further downstream and within the DMPK 3′UTR (FIG. 5). Further, two gRNAs can be administered to the cell: the first targeting sequence between the DMPK stop codon and CTG expansion, and the second targeting sequence downstream of the CTG expansion. The polynucleotides described herein can also be used to modify the DMPK, ATXN8, ATXN8OS, or JPH3 gene.

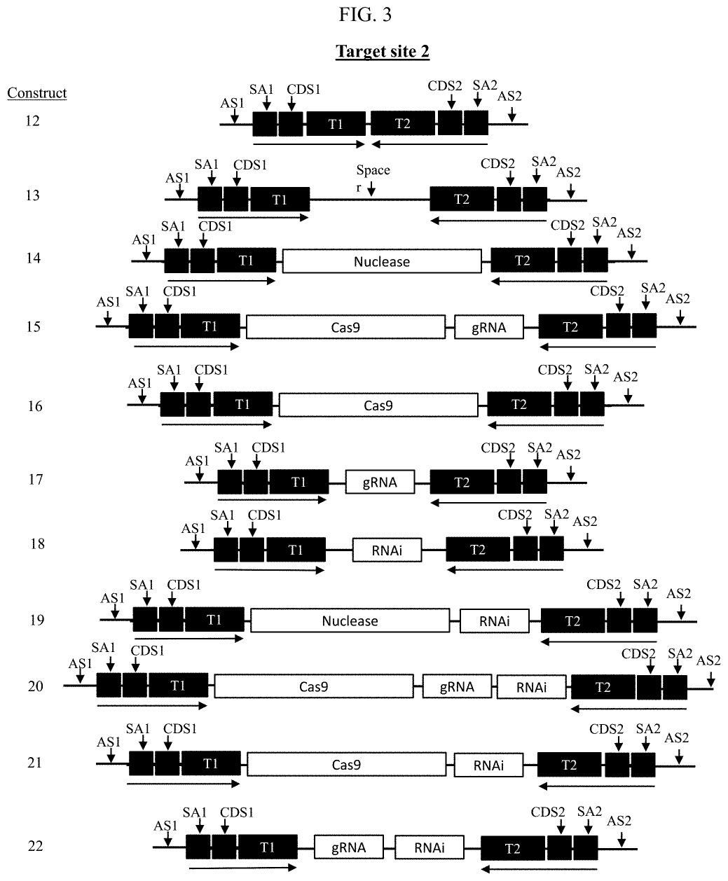

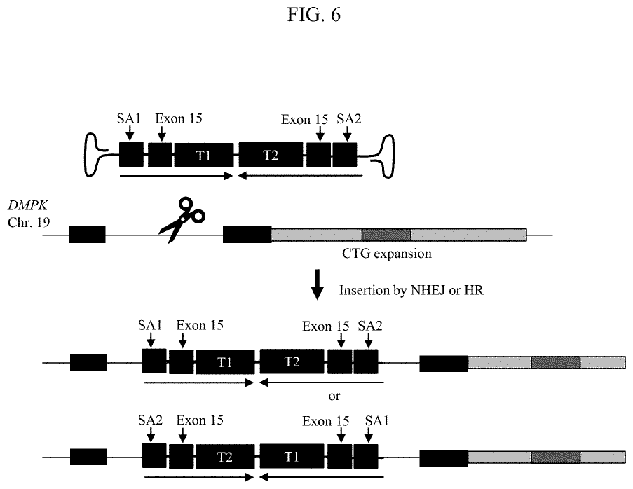

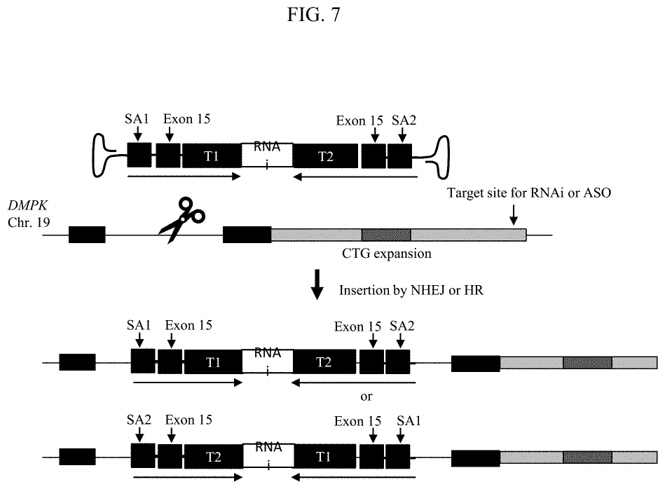

In another embodiment, the methods described herein provide linear polynucleotides for integration into introns of the DMPK gene. The linear polynucleotides can comprise a first splice acceptor operably linked to a first coding sequence, wherein the first coding sequence is also operably linked to a first terminator. The polynucleotide can also comprise a second splice acceptor operably linked to a second coding sequence, wherein the second coding sequence is also operably linked to a second terminator. The two coding sequences can be positioned in a tail-to-tail orientation, with or without a spacer sequence (FIG. 3). The spacer sequence can include functional sequences, including silencing cassettes, rare-cutting endonuclease coding sequences, Cas sequences, or gRNA sequences, or a combination of silencing cassettes, rare-cutting endonuclease coding sequences, Cas sequences, and gRNA sequences. The first and second coding sequences, along with any spacer sequence, can be harbored on a double-stranded or single stranded DNA molecule with exposed DNA ends. The first and second splice acceptors can be the first functional element adjacent to the exposed ends. In other embodiments, the first and second coding sequences, along with any spacer sequence, can be harbored on a viral vector, including an AAV vector with a first and second inverted terminal repeat. Here, first and second splice acceptors can be the first functional element adjacent to the ITRs. In another embodiment, the linear DNA molecules or AAV vectors can also comprise a left and right homology arm. The left and right homology arm can be adjacent to the exposed ends or ITRs, followed by the first and second splice acceptors. The polynucleotides described herein can be used to modify the 3′ end of the DMPK gene (FIG. 1 and FIG. 6). Further, the RNAi comprised within the spacer sequence can target a sequence further downstream and within the 3′ end of the DMPK gene (FIG. 7). Further, two gRNAs can be administered to the cell: the first targeting sequence within an intron and the second targeting sequence downstream of the CTG expansion. The coding sequence can be a partial coding sequence encoding the peptide produced by exon 15 of a wild type DMPK gene.

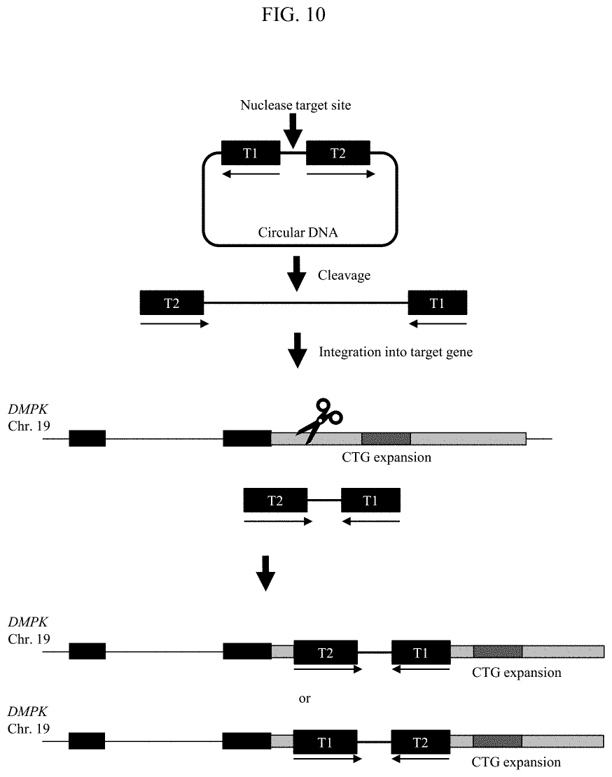

In another embodiment, the methods described herein provide circular polynucleotides for integration into the 3′ UTR of a target gene. The circular polynucleotide can include a first and second terminator having a 5′ and 3′ end and oriented in opposite directions. The polynucleotide can include a first spacer sequence having a cleavage-site for a rare-cutting endonuclease wherein said first spacer sequence is located between the 5′ ends of the first and second terminators (FIG. 10). The polynucleotides can be either double-stranded or single-stranded circular DNA molecules. The polynucleotides can comprise a second spacer sequence located between the 3′ ends of the first and second terminators. The spacer sequence can include functional sequences, including silencing cassettes, rare-cutting endonuclease coding sequences, Cas sequences, or gRNA sequences, or a combination of silencing cassettes, rare-cutting endonuclease coding sequences, Cas sequences, and gRNA sequences. The first and second terminators, along with any spacer sequence, can be harbored on a double-stranded or single stranded DNA molecule with exposed DNA ends. The first and second terminators can be the first functional element adjacent to the exposed ends. In other embodiments, the first and second terminators, along with any spacer sequence, can be harbored on a viral vector, including an AAV vector with a first and second inverted terminal repeat. Here, first and second terminators can be the first functional element adjacent to the ITRs. In another embodiment, the linear DNA molecules or AAV vectors can also comprise a left and right homology arm. The left and right homology arm can be adjacent to the exposed ends or ITRs, followed by the first and second terminators. The polynucleotides described herein can be used to modify the 3′ UTR of the DMPK gene (FIG. 1). Further, the RNAi comprised within the spacer sequence can target a sequence further downstream and within the DMPK 3′UTR. Further, two gRNAs can be administered to the cell: the first targeting sequence between the DMPK stop codon and CTG expansion, and the second targeting sequence downstream of the CTG expansion. The polynucleotides described herein can also be used to modify the DMPK, ATXN8, ATXN8OS, or JPH3 gene.

In another embodiment, the methods described herein provide circular polynucleotides for integration into for integration into introns of the endogenous gene. The polynucleotides can comprise a first splice acceptor operably linked to a first coding sequence operably linked to a terminator, and a second splice acceptor operably linked to a second coding sequence operably linked to a terminator. The polynucleotide can also comprise a cleavage site for a rare-cutting endonuclease, wherein the first splice acceptor operably linked to a first coding sequence operably linked to a terminator is in opposite direction compared to the second splice acceptor operably linked to a second coding sequence operably linked to a terminator, and wherein the cleavage site for the rare-cutting endonuclease is located in a first spacer sequence between the first and second splice acceptors. The first and second coding sequences can encode a full-length protein or a partial protein. The coding sequences can encode the same amino acids, but not the same nucleic acid sequence. The nucleic acid sequence can be different using the degeneracy of the codons. For illustration, the first and second coding sequences can encode the amino acids produced by exon 15 of the DMPK gene and the polynucleotide can be integrated into an intron 14 within the endogenous DMPK gene.

In one embodiment, the polynucleotide can comprise a first and second terminator. The first and second terminators can be positioned within the polynucleotide in opposite directions (i.e., in tail-to-tail orientations). When the polynucleotide is integrated into an endogenous gene in forward or reverse directions, the first and second terminators, terminate transcription from the endogenous gene's promoter. The first and second terminators can be the same terminators or different terminators.

In one embodiment, the polynucleotide can comprise a first and second homology arm flanking the first and second terminator. The first and second homology arms can include sequence that is homologous to a genomic sequence at or near the desired site of integration. The homology arms can be a suitable length for participating in homologous recombination with sequence at or near the desired site of integration. The length of each homology arm can be between 20 nt and 10,000 nt (e.g., 20 nt, 30 nt, 40 nt, 50 nt, 100 nt, 200 nt, 300 nt, 400 nt, 500 nt, 600 nt, 700 nt, 800 nt, 900 nt, 1,000 nt, 2,000 nt, 3,000 nt, 4,000 nt, 5,000 nt, 6,000 nt, 7,000 nt, 8,000 nt, 9,000 nt, 10,000 nt).

In one embodiment, the polynucleotide comprises two splice acceptor sequences, referred to herein as the first and second splice acceptor sequence. The first and second splice acceptor sequences can be positioned within the polynucleotide in opposite directions (i.e., in tail-to-tail orientations) and flanking internal sequences (i.e., coding sequences and terminators). When the polynucleotide is integrated into an intron in forward or reverse directions, the splice acceptor sequences facilitate the removal of the adjacent/upstream intron sequence during mRNA processing. The first and second splice acceptor sequences can be the same sequences or different sequences. One or both splice acceptor sequences can be the splice acceptor sequence of the intron where the polynucleotide is to be integrated. One or both splice acceptor sequences can be a synthetic splice acceptor sequence or a splice acceptor sequence from an intron from a different gene.

In one embodiment, the polynucleotide comprises a first and second coding sequence operably linked to the first and second splice acceptor sequences. The first and second coding sequences are positioned within the polynucleotide in opposite directions (i.e., in tail-to-tail orientations). When the polynucleotide is integrated into an endogenous gene in forward or reverse directions, the first or second coding sequence is transcribed into mRNA by the endogenous gene's promoter. The coding sequences can be designed to correct defective coding sequences, introduce mutations, or introduce novel peptide sequences. The first and second coding sequence can be the same nucleic acid sequence and code for the same protein. Alternatively, the first and second coding sequence can be different nucleic acid sequences and code for the same protein (i.e., using the degeneracy of codons). The coding sequence can encode purification tags (e.g., glutathione-S-transferase, poly(His), maltose binding protein, Strep-tag, Myc-tag, AviTag, HA-tag, or chitin binding protein) or reporter proteins (e.g., GFP, RFP, lacZ, cat, luciferase, puro, neomycin). In one embodiment, the polynucleotide comprises a first and second partial coding sequence operably linked to a first and second splice acceptor sequence, and the polynucleotide does not comprise a promoter.

In some embodiments, the polynucleotides described herein can have a combination of elements including splice acceptors, partial coding sequences, terminators, homology arms, and sites for cleavage by rare-cutting endonucleases. In one embodiment, the combination can be, from 5′ to 3′, [terminator 1]-[terminator 2 RC]. In another embodiment, the combination can be, from 5′ to 3′, [terminator 1]-[gRNA]-[terminator 2 RC]. In another embodiment, the combination can be, from 5′ to 3′, [terminator 1]-[CRISPR enzyme]-[gRNA]-[terminator 2 RC]. In another embodiment, the combination can be, from 5′ to 3′, [splice acceptor 1]-[partial coding sequence 1]-[terminator 1]-[spacer]-[terminator 2 RC]-[partial coding sequence 2 RC]-[splice acceptor 2 RC], where RC stands for reverse complement. This combination can be harbored on a linear DNA molecule or AAV molecule and can be integrated by NHEJ through a targeted break in the target gene. In another embodiment, the polynucleotide can be within a circular DNA molecule and the combination can be, from 5′ to 3′, [rare-cutting endonuclease cleavage site 1]-[splice acceptor 1]-[partial coding sequence 1]-[terminator 1]-[spacer]-[terminator 2 RC]-[partial coding sequence 2 RC]-[splice acceptor 2 RC], wherein the splice acceptor 2 is linked to the rare-cutting endonuclease cleavage site 1.

In another aspect, the polynucleotide for integration can be designed to integrate through multiple repair pathways while creating a desired effect with each outcome. By way of example, a polynucleotide can comprise a first and second terminator, and may be provided to a cell within an AAV genome (i.e., flanked by 145 nucleotide inverted terminal repeats). Following expression by a rare-cutting endonuclease the entire AAV vector can be integrated at the target site by NHEJ in either forward or reverse orientation. Following integration in either the forward or reverse orientation, the endogenous gene can be precisely corrected.

In some embodiments, the location for integration of polynucleotides can be an intron or an intron-exon junction. When targeting an intron, the partial coding sequence can comprise sequence encoding the peptide produced by the following exons within the endogenous gene. For example, if the polynucleotide is designed to be integrated in intron 9 of an endogenous gene with 11 exons, then the partial coding sequence can comprise sequence encoding the peptide produced by exons 10 and 11 of the endogenous gene. When targeting an intron-exon junction, the polynucleotide can be designed to comprise homology arms with sequence homologous to the 3′ of said intron.

In some embodiments, the methods described herein include the use of polynucleotides comprising a first and second coding sequence, wherein both coding sequences encode the same amino acid sequence, and wherein the amino acid sequence is homologous to the protein encoded by the endogenous gene or to a polypeptide fragment thereof. In some embodiments the encoded amino acid sequence encoded by the first and second coding sequence is homologous or identical to a polypeptide fragment of the endogenous gene that is from 5 to 10, from 10 to 20, from 20 to 50, from 50 to 100, from 100 to 200, from 200 to 300, from 300 to 400, from 400 to 500, from 500 to 600, from 600 to 800, from 800 to 1,000, or from 1,000 to 1,200, or more amino acids in length. In some embodiments the encoded amino acid sequence is homologous or identical to a polypeptide fragment of the endogenous gene that is encoded by an exon of the endogenous gene, a partial exon of the endogenous gene, multiple sequential exons of the endogenous gene, a combination of multiple sequential exons and partial exons of the endogenous gene, or the full open reading frame of the endogenous gene. In some embodiments the homology between the amino acid sequence encoded by the first and second coding sequences of the polynucleotide and the protein or fragment thereof that is encoded by the endogenous gene is at least 30%, 40%, 50%, 60%, 70%, 80%, 90%, 95%, or 99%.

In another embodiment, the codon optimization can be split between the first and second partial coding sequences. For example, the first partial coding sequence can have a mixture of non-codon adjusted sequence (i.e., homologous to the corresponding sequence within the endogenous gene-of-interest) and codon adjusted sequence. In this example, the second partial coding sequence can have the opposite adjustment. For example, within a 200 nucleotide partial coding sequence 1 and 2, the nucleotides 1-100 of partial coding sequence 1 can be homologous to the sequence within the endogenous gene-of-interest, and the nucleotides 101-200 can be codon adjusted to have minimal sequence similarities to the endogenous gene-of-interest; the nucleotides 1-100 of partial coding sequence 2 can be codon adjusted to have minimal sequence similarities to the endogenous gene-of-interest, and nucleotides 101-200 can be homologous to the sequence within the endogenous gene-of-interest.

In some embodiments, the polynucleotides described herein can comprise a first and second coding sequence encoding an amino acid sequence that is homologous to an amino acid sequence encoded by an endogenous gene. The coding sequences can be in tail-to-tail orientation and encode the same amino acids. By way of example, a cell can comprise an endogenous gene with 10 exons and 9 introns. A polynucleotide with a first and second coding sequence can encode the amino acids produced by exon 10 of the endogenous gene, and the polynucleotide can be integrated into intron 9. A polynucleotide with a first and second coding sequence can encode the amino acids produced by exon 9 and 10 of the endogenous gene, and the polynucleotide can be integrated into intron 8. A polynucleotide with a first and second coding sequence can encode the amino acids produced by exon 8, 9 and 10 of the endogenous gene, and the polynucleotide can be integrated into intron 7. A polynucleotide with a first and second coding sequence can encode the amino acids produced by exon 7, 8, 9 and 10 of the endogenous gene, and the polynucleotide can be integrated into intron 6. A polynucleotide with a first and second coding sequence can encode the amino acids produced by exon 6, 7, 8, 9 and 10 of the endogenous gene, and the polynucleotide can be integrated into intron 5. A polynucleotide with a first and second coding sequence can encode the amino acids produced by exon 5, 6, 7, 8, 9 and 10 of the endogenous gene, and the polynucleotide can be integrated into intron 4. A polynucleotide with a first and second coding sequence can encode the amino acids produced by exon 4, 5, 6, 7, 8, 9 and 10 of the endogenous gene, and the polynucleotide can be integrated into intron 3. A polynucleotide with a first and second coding sequence can encode the amino acids produced by exon 3, 4, 5, 6, 7, 8, 9 and 10 of the endogenous gene, and the polynucleotide can be integrated into intron 2. A polynucleotide with a first and second coding sequence can encode the amino acids produced by exon 2, 3, 4, 5, 6, 7, 8, 9 and 10 of the endogenous gene, and the polynucleotide can be integrated into intron 1.

In some embodiments, the polynucleotides described herein can comprise a first and second coding sequence encoding the same amino acid sequence but different nucleic acid sequences. Using the degeneracy of codons, the first coding sequence can differ from the second coding sequence. The first coding sequence can have 60%, 70%, 80%, 90%, 95%, or 99% nucleotide homology with the second coding sequence. The first coding sequence can have between 60% to 70%, 70% to 80%, 80% to 90%, 90% to 99% nucleotide homology with the second coding sequence.

In some embodiments, the polynucleotide can comprise a silencing cassette. The silencing cassette can comprise a promoter, a nucleic acid sequence that functions to silence a target nucleic acid, and a terminator. The nucleic acid sequence can be in a format capable of inducing gene silencing within a target nucleic acid (e.g., microRNA, hairpin RNA, antisense RNA). The nucleic acid sequence can be targeted to different regions in the target gene's mRNA, including the 5′ UTR, coding sequence, or 3′ UTR.

In one embodiment, this document features a method of integrating a heterologous polynucleotide into an endogenous gene in the genome of a cell, the method comprising administering to a cell a first recombinant nucleic acid comprising a heterologous polynucleotide comprising in 5′ to 3′ orientation a first terminator and a second terminator in reverse complement, administering to the cell a second recombinant nucleic acid encoding a rare-cutting endonuclease targeted to a site within an endogenous gene in the genome of the cell and/or a gRNA sequence for targeting a rare-cutting endonuclease to a site within an endogenous gene in the genome of the cell, and integrating the heterologous polynucleotide into the endogenous gene at the rare-cutting endonuclease target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene. In one embodiment, the method includes administering the first recombinant nucleic acid at the same time as administering the second recombinant nucleic acid. In another embodiment, the method includes administering the first recombinant nucleic acid before administering the second recombinant nucleic acid. The first recombinant nucleic acid can be administered 1 hour, 2 hours, 4 hours, 8 hours, 12 hours, 1 day, 2 days, 4 days, 8 days, 12 days, 16 days, 20 days, 24 days, or 28 days before the second recombinant nucleic acid. In another embodiment, the method includes administering the second recombinant nucleic acid before administering the first recombinant nucleic acid. The second recombinant nucleic acid can be administered 1 hour, 2 hours, 4 hours, 8 hours, 12 hours, or 1 day before administering the first recombinant nucleic acid.

In one embodiment, this document features a method of integrating a heterologous polynucleotide into an endogenous gene in the genome of a cell, the method comprising administering to a cell a first recombinant nucleic acid comprising a heterologous polynucleotide comprising in 5′ to 3′ orientation a first terminator and a second terminator in reverse complement, administering to the cell a second recombinant nucleic acid encoding a rare-cutting endonuclease targeted to a site within an endogenous gene in the genome of the cell and/or a gRNA sequence for targeting a rare-cutting endonuclease to a site within an endogenous gene in the genome of the cell, and integrating the heterologous polynucleotide into the endogenous gene at the rare-cutting endonuclease target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene. In one embodiment, the first recombinant nucleic acid can be administered with a second recombinant nucleic acid encoding a CRISPR nuclease. In another embodiment, the first recombinant nucleic acid can be administered with a second recombinant nucleic acid encoding a gRNA. In another embodiment, the first recombinant nucleic acid can be administered with a second recombinant nucleic acid encoding a gRNA and a CRISPR nuclease. In another embodiment, the first recombinant nucleic acid can be administered with a second recombinant nucleic acid encoding a zinc-finger nuclease. In another embodiment, the first recombinant nucleic acid can be administered with a second recombinant nucleic acid encoding a meganuclease nuclease. In another embodiment, the first recombinant nucleic acid can be administered with a second recombinant nucleic acid encoding a TALE nuclease. In another embodiment, the first recombinant nucleic acid can be administered with a second recombinant nucleic acid encoding a zinc-finger nuclease and CRISPR nuclease. In another embodiment, the first recombinant nucleic acid can be administered with a second recombinant nucleic acid encoding a TALE nuclease and CRISPR nuclease. In another embodiment, the first recombinant nucleic acid can be administered with a second recombinant nucleic acid encoding a meganuclease and CRISPR nuclease. In another embodiment, the first recombinant nucleic acid can be administered with a second recombinant nucleic acid encoding a meganuclease and TALE nuclease. In another embodiment, the first recombinant nucleic acid can be administered with a second recombinant nucleic acid encoding a meganuclease and zinc-finger nuclease.

In one embodiment, this document features a method of integrating a heterologous polynucleotide into an endogenous gene in the genome of a cell, the method comprising administering to a cell a first recombinant nucleic acid comprising a heterologous polynucleotide comprising in 5′ to 3′ orientation a first terminator and a second terminator in reverse complement, administering to the cell a second recombinant nucleic acid encoding a CRISPR protein, and administering a third recombinant nucleic acid encoding a gRNA sequence for targeting a CRISPR protein to a site within an endogenous gene in the genome of the cell, and integrating the heterologous polynucleotide into the endogenous gene at the gRNA target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene.

In one embodiment, this document provides a method of integrating a heterologous polynucleotide into an endogenous gene in the genome of a cell, the method comprising administering to a cell a recombinant nucleic acid comprising a heterologous polynucleotide comprising in 5′ to 3′ orientation a first terminator, a sequence encoding a rare-cutting endonuclease targeted to a site within an endogenous gene in the genome of the cell, and a second terminator in reverse complement, integrating the heterologous polynucleotide into the endogenous gene at the rare-cutting endonuclease target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene. In one embodiment, the rare-cutting endonuclease can be a CRISPR nuclease. In another embodiment, the rare-cutting endonuclease can be a meganuclease nuclease. In another embodiment, the rare-cutting endonuclease can be a TALE nuclease. In another embodiment, the rare-cutting endonuclease can be a zinc-finger nuclease. In another embodiment, the rare-cutting endonuclease can be a CRISPR protein and a gRNA sequence.

In one embodiment, this document provides a method of integrating a heterologous polynucleotide into an endogenous gene in the genome of a cell, the method comprising administering to a cell a recombinant nucleic acid comprising a heterologous polynucleotide comprising in 5′ to 3′ orientation a first terminator, a sequence encoding a CRISPR protein, and a second terminator in reverse complement, administering to the cell a nucleic acid encoding a gRNA for targeting a CRISPR protein to an endogenous gene in the genome of the cell, and integrating the heterologous polynucleotide into the endogenous gene at the gRNA target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene. In one embodiment, the sequence encoding a CRISPR protein can be, from 5′ to 3′, [promoter]-[CRISPR protein]-[terminator]. In another embodiment, the sequence encoding a CRISPR protein can be, from 5′ to 3′, [terminator RC]-[CRISPR protein RC]-[promoter RC].

In one embodiment, this document provides a method of integrating a heterologous polynucleotide into an endogenous gene in the genome of a cell, the method comprising administering to a cell a recombinant nucleic acid comprising a heterologous polynucleotide comprising in 5′ to 3′ orientation a first terminator, a sequence encoding a rare-cutting endonuclease targeted to a site within an endogenous gene in the genome of the cell, and a second terminator in reverse complement, integrating the heterologous polynucleotide into the endogenous gene at the rare-cutting endonuclease target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene. In one embodiment, the sequence encoding the rare-cutting endonuclease is, from 5′ to 3′, [promoter]-[rare-cutting endonuclease]-[terminator]. In another embodiment, the sequence encoding the rare-cutting endonuclease is, from 5′ to 3′, [terminator RC]-[rare-cutting endonuclease RC]-[promoter RC], where RC is reverse complement. In one embodiment, the rare-cutting endonuclease sequence can be a CRISPR/Cas nuclease and can be, from 5′ to 3′, [promoter]-[Cas9]-[terminator]-[gRNA promoter]-[gRNA]-[gRNA terminator]. In another embodiment, the rare-cutting endonuclease sequence can be a CRISPR/Cas nuclease and can be, from 5′ to 3′, [gRNA terminator RC]-[gRNA RC]-[gRNA promoter RC]-[terminator RC]-[Cas9 RC]-[promoter RC]. In another embodiment, the rare-cutting endonuclease sequence can be a CRISPR/Cas nuclease and can be, from 5′ to 3′, [gRNA promoter]-[gRNA]-[gRNA terminator]-[promoter]-[Cas9]-[terminator]. In another embodiment, the rare-cutting endonuclease sequence can be a CRISPR/Cas nuclease and can be, from 5′ to 3′, [terminator RC]-[Cas9 RC]-[promoter RC]-[gRNA terminator RC]-[gRNA RC]-[gRNA promoter RC]. In one embodiment, this document provides a method of integrating a heterologous polynucleotide into an endogenous gene in the genome of a cell, the method comprising administering to a cell a recombinant nucleic acid comprising a heterologous polynucleotide comprising in 5′ to 3′ orientation a first terminator, a sequence encoding a gRNA for targeting a CRISPR protein to a site within an endogenous gene in the genome of the cell, and a second terminator in reverse complement, and administering to the cell a CRISPR protein or a nucleic acid encoding a CRISPR protein, and integrating the heterologous polynucleotide into the endogenous gene at the gRNA target site to provide a modified endogenous gene in which the first terminator or the second terminator is operatively linked to a promoter of the endogenous gene, wherein the modified endogenous gene produces an mRNA transcript that is truncated relative to an mRNA transcript produced by the endogenous gene. In one embodiment, the gRNA can be, in 5′ to 3′, [gRNA promoter]-[gRNA]-[gRNA terminator]. In another embodiment, the gRNA can be in 5′ to 3′, [gRNA terminator RC]-[gRNA RC]-[gRNA promoter RC].

In one embodiment, this document features a polynucleotide comprising a first and second terminator in a tail-to-tail orientation, wherein the polynucleotide does not comprise a coding sequence operably linked to the first terminator and does not comprise a coding sequence operably linked to the second terminators. The polynucleotide can be a non-naturally occurring polynucleotide with two terminators in tail-to-tail orientation. The position of the two terminators in tail-to-tail orientation can result in a polynucleotide that is not found in nature. The two terminators can be the same terminators, for example, two SV40 terminators, or they can be different terminators, for example, an SV40 terminator and a BGH terminator. The sequence encoding the two terminators can be chemically synthesized within a vector with a selectable marker and origin of replication for bacterial cloning. The vector can include, but not limited to, pUC19, pBR237, pBR322, pET3a, pBluescript II KS (+), pEXP4-DEST, pSP72, pET SUMO, pCR 2.1-TOPO, pBAD TOPO, pGEX-4T2, pQE-30, or pACYC177. The vector comprising the polynucleotide with two terminators can be isolated and purified from bacteria and stored at 4 Celsius, −20 Celsius, or −80 Celsius.

The methods and compositions provided herein can be used within to modify endogenous genes within cells. The endogenous genes can include, fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, glucocerebrosidase (GBA), α-galactosidase A (GLA), iduronate sulfatase (IDS), iduronidase (IDUA), acid sphingomyelinase (SMPD1), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARG1 (arginase), and/or a solute carrier family 25 (SLC25A13, an aspartate/glutamate carrier) protein, a UGT1A1 or UDP glucuronsyltransferase polypeptide A1, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, an USH2A protein, an ATXN protein, and a lipoprotein lyase (LPL) protein.

In some embodiments, the polynucleotides described herein comprising a silencing cassette can be used to correct gain-of-function disorders by silencing specific genes and replacing the expression of the genes. The genes can include SODI, TRPV4, CHRNA1, CHRND, CHRNE, CHRNB1, PRPS1, LRRK2, STIM1, FGFR3, MECP2, SNCA, ATXN1, ATXN2, ATXN3, CACNA1A, ATXN7, TBP, HTT, JPH3, AR, FXN, DMPK, PABPN1, ATXN8, ATXN8OS, RHO, and C9orf72.

The polynucleotide may include sequence for modifying the sequence encoding a polypeptide that is lacking or non-functional or having a gain-of-function mutation in the subject having a genetic disease, including but not limited to the following genetic diseases: achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, pert syndrome, arrhythmogenic right ventricular dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

Additional diseases that can be treated by targeted integration include von Willebrand disease, usher syndrome, polycystic kidney disease, spinocerebellar ataxia type 3, and spinocerebellar ataxia type 6.

As described herein, the donor molecule can be in a viral or non-viral vector. The vectors can be in the form of circular or linear double-stranded or single stranded DNA. The donor molecule can be conjugated or associated with a reagent that facilitates stability or cellular update. The reagent can be lipids, calcium phosphate, cationic polymers, DEAE-dextran, dendrimers, polyethylene glycol (PEG) cell penetrating peptides, gas-encapsulated microbubbles or magnetic beads. The donor molecule can be incorporated into a viral particle. The virus can be retroviral, adenoviral, adeno-associated vectors (AAV), herpes simplex, pox virus, hybrid adenoviral vector, epstein-bar virus, lentivirus, or herpes simplex virus.

In some embodiments, the AAV vectors as described herein can be derived from any AAV. In some embodiments, the AAV vector is derived from the defective and nonpathogenic parvovirus adeno-associated type 2 virus. All such vectors are derived from a plasmid that retains only the AAV 145 bp inverted terminal repeats flanking the polynucleotide expression cassette. Efficient gene transfer and stable polynucleotide delivery due to integration into the genomes of the transduced cell are key features for this vector system. (Wagner et al., Lancet 351:9117 1702-3, 1998; Kearns et al., Gene Ther. 9:748-55, 1996). Other AAV serotypes, including AAV1, AAV2, AAV3, AAV4, AAV5, AAV6, AAV7, AAV8, AAV9 and AAVrh.10 and any novel AAV serotype can also be used in accordance with the present invention. In some embodiments, chimeric AAV is used where the viral origins of the long terminal repeat (LTR) sequences of the viral nucleic acid are heterologous to the viral origin of the capsid sequences. Non-limiting examples include chimeric virus with LTRs derived from AAV2 and capsids derived from AAV5, AAV6, AAV8 or AAV9 (i.e. AAV2/5, AAV2/6, AAV2/8 and AAV2/9, respectively).

The constructs described herein may also be incorporated into an adenoviral vector system. Adenoviral based vectors are capable of very high transduction efficiency in many cell types and do not require cell division.

In other embodiments, the polynucleotides described herein can be delivered by non-viral mechanisms, including magnetic nanoparticles or lipid nanoparticles. In an embodiment, the polynucleotides delivered with lipid nanoparticles. As used herein, the term “lipid nanoparticle” refers to a transfer vehicle comprising one or more lipids. The term “lipid nanoparticle” also refers to particles having at least one dimension on the order of nanometers (e.g., 1-1,000 nm) which include one or more of the compounds of formula (I) or other specified cationic lipids. The one or more lipids can be cationic lipids, non-cationic lipids, or PEG-modified lipids. The lipid nanoparticles can be formulated to deliver one or more gene editing reagents to one or more target cells. Examples of suitable lipids include phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a gene editing reagent to a target cell.

In an embodiment, this document describes the use of lipid nanoparticles as transfer vehicles comprising a cationic lipid to encapsulate and/or enhance the delivery of a gene editing reagent into a target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. In some embodiments, the compositions and methods within this document employ lipid nanoparticles comprising (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), or (15Z,18Z)—N,N-dimethyl-6-((9Z,12Z)-octadeca-9,12-dien-1-yl)tetracosa-5,15,18-trien-1-amine (HGT5002).

In an embodiment, the gene editing reagents can be delivered with the lipid nanoparticle BAMEA-016B. The gene editing reagents can be in the form of RNA or DNA. For example, the gene editing reagents can be Cas9 mRNA and sgRNA combined with BAMEA-016B lipid nanoparticles.

In some embodiments, the cationic lipid N-[1-(2,3-dioleyloxy)propyl]-N,N,N-trimethylammonium chloride (DOTMA) can be used. DOTMA can be formulated alone or combined with the neutral lipid, dioleoylphosphatidyl-ethanolamine (DOPE) or other cationic or non-cationic lipids into a liposomal transfer vehicle or a lipid nanoparticle, and such liposomes can be used to enhance the delivery of nucleic acids into target cells. Other suitable cationic lipids include, 5-carboxyspermylglycinedioctadecylamide,” 2,3-dioleyloxy-N-[2(spermine-carboxamido)ethyl]-N,N-dimethyl-1-propanaminium, 1,2-Dioleoyl-3-Dimethylammonium-Propane, 1,2-Dioleoyl-3-Trimethylammonium-Propane. Contemplated cationic lipids also include 1,2-distearyloxy-N,N-dimethyl-3-aminopropane, 1,2-dioleyloxy-N,N-dimethyl-3-aminopropane, 1,2-dilinoleyloxy-N,N-dimethyl-3-aminopropane, 1,2-dilinolenyloxy-N,N-dimethyl-3-aminopropane, N-dioleyl-N,N-dimethylammonium chloride, N,N-distearyl-N,N-dimethylammonium bromide, N-(1,2-dimyristyloxyprop-3-yl)-N,N-dimethyl-N-hydroxyethyl ammonium bromide, 3-dimethylamino-2-(cholest-5-en-3-beta-oxybutan-4-oxy)-1-(cis,cis-9,12-octadecadienoxy)propane, 2-[5′-(cholest-5-en-3-beta-oxy)-3′-oxapentoxy)-3-dimethyl-1-(cis,cis-9′,1-2′-octadecadienoxy)propane, N,N-dimethyl-3,4-dioleyloxybenzylamine, 1,2-N,N′-dioleylcarbamyl-3-dimethylaminopropane, 2,3-Dilinoleoyloxy-N,N-dimethylpropylamine, 1,2-N,N′-Dilinoleylcarbamyl-3-dimethylaminopropane, 1,2-Dilinoleoylcarbamyl-3-dimethylaminopropane, 2,2-dilinoleyl-4-dimethylaminomethyl-[1,3]-dioxolane, 2,2-dilinoleyl-4-dimethylaminoethyl-[1,3]-dioxolane, and 2-(2,2-di((9Z,12Z)-octadeca-9,12-dien-1-yl)-1,3-dioxolan-4-yl)-N,N-dimethylethanamine (DLin-KC2-DMA)), or mixtures thereof.

In some embodiments, cholesterol-based cationic lipids can be used to facilitate delivery of gene editing reagents to target cells in the present document. Cholesterol-based cationic lipids can be used alone or in combination with other cationic or non-cationic lipids. Suitable cholesterol-based cationic lipids include DC-Chol (N,N-dimethyl-N-ethylcarboxamidocholesterol), or 1,4-bis(3-N-oleylamino-propyl)piperazine.

In some embodiments, cationic lipids such as the dialkylamino-based, imidazole-based, and guanidinium-based lipids are used to facilitate delivery of gene editing reagents to target cells in the present document. For example, certain embodiments are directed to a composition comprising one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate.

The imidazole-based cationic lipids are also characterized by their reduced toxicity relative to other cationic lipids. The imidazole-based cationic lipids (e.g., ICE) may be used as the sole cationic lipid in the lipid nanoparticle, or alternatively may be combined with traditional cationic lipids, non-cationic lipids, and PEG-modified lipids. The cationic lipid may comprise a molar ratio of about 1% to about 90%, about 2% to about 70%, about 5% to about 50%, about 10% to about 40% of the total lipid present in the transfer vehicle, or preferably about 20% to about 70% of the total lipid present in the transfer vehicle.

In other embodiments the gene editing reagents and methods described herein are use lipid nanoparticles comprising one or more cleavable lipids, such as, for example, one or more cationic lipids or compounds that comprise a cleavable disulfide (S—S) functional group (e.g., HGT4001, HGT4002, HGT4003, HGT4004 and HGT4005).

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipids together which comprise the transfer vehicle (e.g., a lipid nanoparticle). Contemplated PEG-modified lipids include, but is not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target cell, or they may be selected to rapidly exchange out of the formulation in vivo. Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

The present document also contemplates the use of non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the transfer vehicle.

In one embodiment, the lipid nanoparticle is prepared by combining multiple lipid and/or polymer components. For example, a transfer vehicle may be prepared using C12-200, DOPE, chol, DMG-PEG2K at a molar ratio of 40:30:25:5, or DODAP, DOPE, cholesterol, DMG-PEG2K at a molar ratio of 18:56:20:6, or HGT5000, DOPE, chol, DMG-PEG2K at a molar ratio of 40:20:35:5, or HGT5001, DOPE, chol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). The molar ratios may be adjusted accordingly. For example, In some embodiments, In some embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

In some embodiments, the lipid nanoparticles can comprise at least one of the following cationic lipids: C12-200, DLin-KC2-DMA, DODAP, HGT4003, ICE, HGT5000, or HGT5001. In some embodiments, the transfer vehicle comprises cholesterol and/or a PEG-modified lipid. In some embodiments, the transfer vehicles comprise DMG-PEG2K. In some embodiments, the transfer vehicle comprises one of the following lipid formulations: C12-200, DOPE, DMG-PEG2K; DODAP, DOPE, cholesterol, DMG-PEG2K; HGT5000, DOPE, DMG-PEG2K, HGT5001, DOPE, DMG-PEG2K.

The liposomal transfer vehicles for use with the gene editing reagents of the invention can be prepared by various techniques. For example, multi-lamellar vesicles (MLV) are prepared by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

Liposomal transfer vehicles may be designed according to delivering gene editing reagents to target organs. For example, to target hepatocytes in the liver, a liposomal transfer vehicle may be sized such that its dimensions are smaller than the fenestrations of the endothelial layer lining within the liver. In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

The methods and compositions described herein are applicable to any eukaryotic organism in which it is desired to alter the organism through genomic modification. The eukaryotic organisms include plants, algae, animals, fungi and protists. The eukaryotic organisms can also include plant cells, algae cells, animal cells, fungal cells and protist cells.

Exemplary mammalian cells include, but are not limited to, oocytes, K562 cells, CHO (Chinese hamster ovary) cells, HEP-G2 cells, BaF-3 cells, Schneider cells, COS cells (monkey kidney cells expressing SV40 T-antigen), CV-1 cells, HuTu80 cells, NTERA2 cells, NB4 cells, HL-60 cells and HeLa cells, 293 cells (see, e.g., Graham et al. (1977) J. Gen. Virol. 36:59), and myeloma cells like SP2 or NSO (see, e.g., Galfre and Milstein (1981) Meth. Enzymol. 73(B):3 46). Peripheral blood mononucleocytes (PBMCs) or T-cells can also be used, as can embryonic and adult stem cells. For example, stem cells that can be used include embryonic stem cells (ES), induced pluripotent stem cells (iPSC), mesenchymal stem cells, hematopoietic stem cells, liver stem cells, skin stem cells and neuronal stem cells.

The methods and compositions of the invention can be used in the production of modified organisms. The modified organisms can be small mammals, companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. The methods and compositions of the invention can be used in humans.

Exemplary plants and plant cells which can be modified using the methods described herein include, but are not limited to, monocotyledonous plants (e.g., wheat, maize, rice, millet, barley, sugarcane), dicotyledonous plants (e.g., soybean, potato, tomato, alfalfa), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); vegetative crops for consumption (e.g. soybean and other legumes, squash, peppers, eggplant, celery etc), flowering plants (e.g., petunia, rose, chrysanthemum), conifers and pine trees (e.g., pine fir, spruce); poplar trees (e.g. P. tremula×P. alba); fiber crops (cotton, jute, flax, bamboo) plants used in phytoremediation (e.g., heavy metal accumulating plants); oil crops (e.g., sunflower, rape seed) and plants used for experimental purposes (e.g., Arabidopsis). The methods disclosed herein can be used within the genera Asparagus, Avena, Brassica, Citrus, Citrullus, Capsicum, Cucurbita, Daucus, Erigeron, Glycine, Gossypium, Hordeum, Lactuca, Lolium, Lycopersicon, Malus, Manihot, Nicotiana, Orychophragmus, Oryza, Persea, Phaseolus, Pisum, Pyrus, Prunus, Raphanus, Secale, Solanum, Sorghum, Triticum, Vitis, Vigna, and Zea. The term plant cells include isolated plant cells as well as whole plants or portions of whole plants such as seeds, callus, leaves, and roots. The present disclosure also encompasses seeds of the plants described above wherein the seed has the has been modified using the compositions and/or methods described herein. The present disclosure further encompasses the progeny, clones, cell lines or cells of the transgenic plants described above wherein said progeny, clone, cell line or cell has the polynucleotide or gene construct. Exemplary algae species include microalgae, diatoms, Botryococcus braunii, Chlorella, Dunaliella tertiolecta, Gracileria, Pleurochrysis carterae, Sorgassum and Ulva.

The methods described in this document can include the use of rare-cutting endonucleases for stimulating homologous recombination or non-homologous integration of a polynucleotide molecule into an endogenous gene. The rare-cutting endonuclease can include CRISPR, TALENs, or zinc-finger nucleases (ZFNs). The CRISPR system can include CRISPR/Cas9 or CRISPR/Cas12a (Cpf1). The CRISPR system can include variants which display broad PAM capability (Hu et al., Nature 556, 57-63, 2018; Nishimasu et al., Science DOI: 10.1126, 2018) or higher on-target binding or cleavage activity (Kleinstiver et al., Nature 529:490-495, 2016). The gene editing reagent can be in the format of a nuclease (Mali et al., Science 339:823-826, 2013; Christian et al., Genetics 186:757-761, 2010), nickase (Cong et al., Science 339:819-823, 2013; Wu et al., Biochemical and Biophysical Research Communications 1:261-266, 2014), CRISPR-FokI dimers (Tsai et al., Nature Biotechnology 32:569-576, 2014), or paired CRISPR nickases (Ran et al., Cell 154:1380-1389, 2013).

The methods and compositions described in this document can be used in a circumstance where it is desired to modify the 3′ UTR of an endogenous gene. For example, patients with myotonic dystrophy type 1 have a CTG repeat expansion in the DMPK 3′ UTR. These patients may benefit from integration of the polynucleotides described herein into the 3′ UTR or the upstream introns. Further, the methods described herein may be useful for delivery of donor molecules using viral or non-viral mechanisms. The methods described herein can also be used in combination with RNAi and other genome editing methods. For example, a polynucleotide comprising two terminators for integration into the 3′ UTR of the DMPK gene can be combined with RNAi targeting the 3′ UTR downstream of the terminators and a second gRNA that targets sequence downstream of the CTG expansion. The resulting edits can be one or a combination of i) silencing of the mutant allele by RNA, ii) insertion of the first terminator to prevent transcription of the CTG expansion, iii) insertion of the second terminator to prevent transcription of the CTG expansion, or iv) removal of the expanded CTG expansion by two cuts by the rare-cutting endonuclease.

In one embodiment, the polynucleotides provided herein result in reduced levels or expression of toxic mRNA transcripts. For example, if a polynucleotide comprising two terminators is integrated into the 3′ UTR of the DMPK gene, then the levels of DMPK transcripts comprising repeat expansion sequences can be reduced. The polynucleotide can reduce the levels of DMPK transcripts comprising repeat expansion sequences within a cell by about 10%, about 20%, about 30%, about 40%, about 50%, about 60%, about 70%, about 80%, about 90%, about 95%, about 99%, compared to a cell that is not administered the polynucleotide. The polynucleotide can reduce the levels of DMPK transcripts comprising repeat expansion sequences within a cell by about 5-10%, about 10-20%, about 20-30%, about 30-40%, about 40-50%, about 50-60%, about 60-70%, about 70-80%, about 80-90%, about 90-95%, about 95-99%, compared to a cell that is not administered the polynucleotide. The polynucleotide can reduce the levels of DMPK transcripts comprising repeat expansion sequences within a cell by greater than 10%, greater than 20%, greater than 30%, greater than 40%, greater than 50%, greater than 60%, greater than 70%, greater than 80%, greater than 90%, greater than 95%, greater than 99%, compared to a cell that is not administered the polynucleotide.

In one embodiment, the polynucleotides provided herein result in truncated mRNA transcripts with reduced levels of mRNA with repeat expansion sequences. For example, if a polynucleotide comprising two terminators is integrated into the 3′ UTR of the DMPK gene, then the levels of truncated DMPK transcripts increase relative to a cell that is not delivered the polynucleotide, and the levels of mRNA with repeat expansion sequences reduces relative to a cell that is not delivered the polynucleotide. The polynucleotide can reduce the levels of DMPK transcripts comprising repeat expansion sequences within a cell by about 10%, about 20%, about 30%, about 40%, about 50%, about 60%, about 70%, about 80%, about 90%, about 95%, about 99%, compared to a cell that is not administered the polynucleotide. The polynucleotide can reduce the levels of DMPK transcripts comprising repeat expansion sequences within a cell by about 5-10%, about 10-20%, about 20-30%, about 30-40%, about 40-50%, about 50-60%, about 60-70%, about 70-80%, about 80-90%, about 90-95%, about 95-99%, compared to a cell that is not administered the polynucleotide. The polynucleotide can reduce the levels of DMPK transcripts comprising repeat expansion sequences within a cell by greater than 10%, greater than 20%, greater than 30%, greater than 40%, greater than 50%, greater than 60%, greater than 70%, greater than 80%, greater than 90%, greater than 95%, greater than 99%, compared to a cell that is not administered the polynucleotide.

The methods described herein can have benefits for treating repeat expansion diseases over conventional approaches. As described above, the methods include integrating a polynucleotide into an endogenous gene comprising a gain-of-function or repeat expansion mutation. One benefit provided by the methods described herein includes high-efficacy correction of the phenotype. The use of a polynucleotide with two terminators can increase the number of cells that exhibit a corrected phenotype (i.e., reduced toxic mRNA or protein), compared to using traditional gene editing approaches (i.e., using traditional HR-based uni-directional templates). The relative increase in the number cells comprising reduced toxic mRNA or protein can be about 1.5 times, 2 times, 2.5 times, or more when compared to the number of cells delivered traditional HR-based uni-directional templates.

The invention will be further described in the following examples, which do not limit the scope of the invention described in the claims.

### EXAMPLES

### Example 1: Design of Polynucleotides for Targeted Integration of Bi-Directional Terminators into DMPK

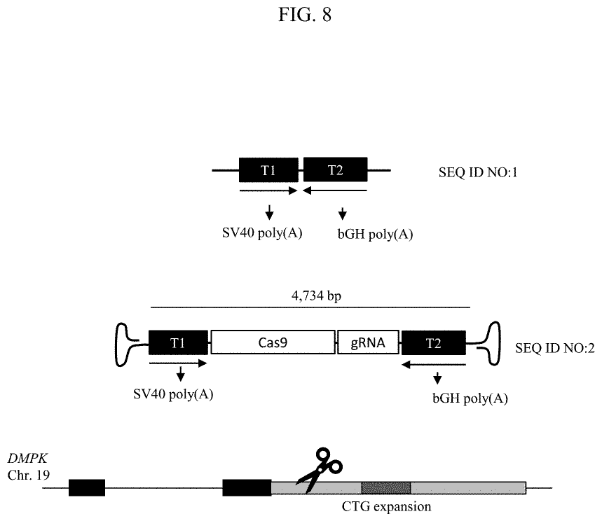

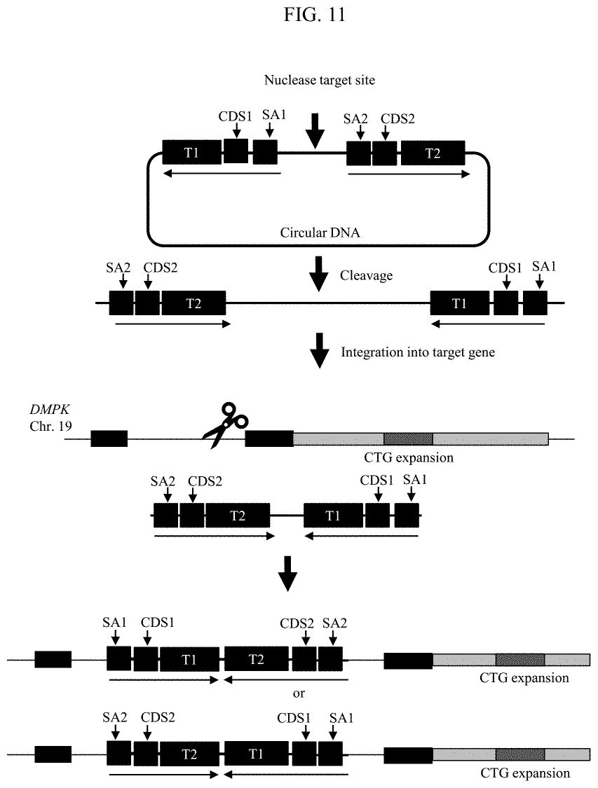

Four polynucleotides are designed, each comprising two terminators. The first polynucleotide is in the format of double-stranded, linear DNA, and comprises an SV40 poly(A) sequence followed by a bGH poly(A) sequence in the reverse direction (SEQ ID NO:1; FIG. 8). The second polynucleotide is harbored on an AAV vector and comprises the same SV40 poly(A) sequence and bGH poly(A) sequence; however, a CMV:SaCas9 and gRNA sequence is placed between the two terminators (SEQ ID NO:2; FIG. 8). The third vector is harbored on plasmid DNA and comprises a SV40 poly(A) sequence and bGH poly(A) sequence, wherein the additional plasmid DNA sequences are harbored between the two terminators, and wherein a target site for a rare-cutting endonuclease is present between the terminators (FIG. 10). The fourth vector comprises the same sequence as the third vector, however, the terminators were operably linked to a splice acceptor and coding sequence encoding exon 15 of the DMPK gene (FIG. 11).

### Example 2: Design of Nucleases for Targeting the 3′ End of the DMPK Gene

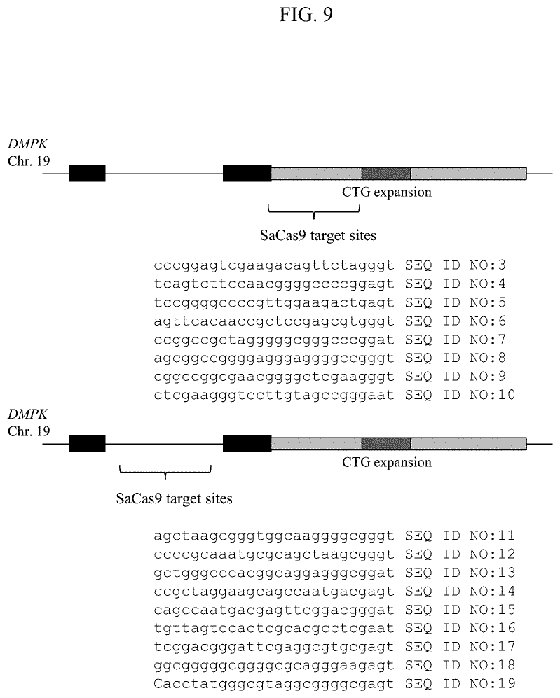

Two sets of gRNAs are designed, the first targeting sequence within the 3′ UTR of the DMPK gene and the second targeting sequence within intron 14 of the DMPK gene (FIG. 9). The gRNAs targeting the 3′ UTR of the DMPK gene are administered to cells with the polynucleotides comprising two terminators that are not operably linked to any coding sequences. The gRNAs targeting intron 14 of the DMPK gene are administered to cells with the polynucleotides comprising exon 14 DMPK coding sequence operably linked to the two terminators. The gRNAs for targeting the 3′ UTR include CCCGGAGTCGAAGACAGTTCTAGGGT (SEQ ID NO:3) TCAGTCTTCCAACGGGGCCCCGGAGT (SEQ ID NO:4) TCCGGGGCCCCGTTGGAAGACTGAGT (SEQ ID NO: 5) AGTTCACAACCGCTCCGAGCGTGGGT (SEQ ID NO:6) CCGGCCGCTAGGGGGCGGGCCCGGAT (SEQ ID NO:7) AGCGGCCGGGGAGGGAGGGGCCGGGT (SEQ ID NO:8) CGGCCGGCGAACGGGGCTCGAAGGGT (SEQ ID NO:9) and CTCGAAGGGTCCTTGTAGCCGGGAAT (SEQ ID NO:10). The gRNAs targeting intron 14 include AGCTAAGCGGGTGGCAAGGGGCGGGT (SEQ ID NO:11), CCCCGCAAATGCGCAGCTAAGCGGGT (SEQ ID NO:12), GCTGGGCCCACGGCAGGAGGGCGGAT (SEQ ID NO: 13), CCGCTAGGAAGCAGCCAATGACGAGT (SEQ ID NO:14), CAGCCAATGACGAGTTCGGACGGGAT (SEQ ID NO: 15), TGTTAGTCCACTCGCACGCCTCGAAT (SEQ ID NO: 16), TCGGACGGGATTCGAGGCGTGCGAGT (SEQ ID NO: 17), GGCGGGGGCGGGGCGCAGGGAAGAGT (SEQ ID NO:18), and CACCTATGGGCGTAGGCGGGGCGAGT (SEQ ID NO: 19).

### Example 3: Integrating Polynucleotides with Bi-Directional Terminators into the DMPK Gene

Transfection is performed using HEK293 cells. HEK293 cells are maintained at 37° C. and 5% CO2 in DMEM high supplemented with 10% fetal bovine serum (FBS). HEK293T cells are transfected with the polynucleotide and CRISPR constructs. Transfections are performed using lipofection. Genomic DNA and RNA is isolated 72 hours post transfection. Genomic DNA is assessed for integration events, and RNA is assessed for levels of DMPK transcripts comprising CUG repeat sequences and transcripts not comprising the CUG repeat sequences.

### Example 4: Design of Polynucleotides for Targeted Integration of Bi-Directional Terminators into ATXN8

Three polynucleotides are designed, each comprising two terminators. The first polynucleotide is in the format of double-stranded, linear DNA, and comprises an SV40 poly(A) sequence followed by a bGH poly(A) sequence in the reverse direction (SEQ ID NO:1). The second polynucleotide is harbored on an AAV vector and comprises the same SV40 poly(A) sequence and bGH poly(A) sequence; however, a CMV:SaCas9 and gRNA sequence is placed between the two terminators (SEQ ID NO:2). The third vector is harbored on plasmid DNA and comprises a SV40 poly(A) sequence and bGH poly(A) sequence, wherein the additional plasmid DNA sequences are harbored between the two terminators, and wherein a target site for a rare-cutting endonuclease is present between the terminators (FIG. 10).

### Example 5: Design of Nucleases for Targeting the ATXN8 Gene

Nucleases are designed to target sequence within the ATXN8 gene, and upstream of the CTG/CAG trinucleotide repeat expansion on chromosome 13q21.

### Example 6: Integrating Bi-Directional Terminators into the ATXN8 Gene

Transfection is performed using HEK293 cells. HEK293 cells are maintained at 37° C. and 5% CO2 in DMEM high supplemented with 10% fetal bovine serum (FBS). HEK293T cells are transfected with the polynucleotides and CRISPR constructs. Transfections are performed using lipofection. Genomic DNA and RNA is isolated 72 hours post transfection. Genomic DNA is assessed for integration events, and RNA is assessed for levels of transcripts comprising CUG/CAG repeat sequences and transcripts not comprising the CUG/CAG repeat sequences.

### Example 7: Integrating Bi-Directional Terminators into DMPK In Vivo

Polynucleotides comprising two terminators are delivered to muscle cells in vivo using AAV. Different AAV vectors are designed, each comprising two terminators. The first AAV vector comprises two terminators in tail-to-tail orientation with a CMV:SaCas9 and U6-gRNA sequence between the two terminators. The SaCas9 nuclease targets sequence within the 3′ UTR of the DMPK gene. The second AAV vector comprises a splice acceptor operably linked to a coding sequence for exon 14 of the DMPK gene, operably linked to a terminator. This sequence is placed in tail-to-tail orientation with a second splice acceptor operably linked to a second coding sequence for exon 14 of the DMPK gene, operably linked to a second terminator. This AAV vector is administered together with a second AAV vector comprising a CMV:SaCas9 and U6-gRNA sequence. The third AAV vector comprises two terminators in tail-to-tail orientation with an shRNA silencing cassette targeting mRNA sequence downstream of the CUG repeat sequence. This AAV vector is administered together with a second AAV vector comprising a CMV:SaCas9 and U6-gRNA sequence.

AAV comprising the polynucleotides with two terminators are applied systemically in C57BL/6 using the muscle-tropic rAAV serotype 6. 4-week-old mice are injected via tail-vein injection. Mice are administered 4E+12 vector genomes of each bi-directional AAV vector.

8-weeks post injection, genomic DNA and total RNA is extracted from TA muscles. To evaluate the level of reduction of transcripts with the CUG repeat sequence, quantitative PCR is performed on the RNA. To evaluate successful targeted insertion, the genomic DNA is used for PCR to detect the 5′ and 3′ junction of the insertion events with the bi-directional AAV vectors.

SEQ ID NO: 1
Aacttgtttattgcagcttataatggttacaaataaagcaatagcatcacaaatttcacaaataaagcatttttttcactgcattct
agttgtggtttgtccaaactcatcaatgtatcttatcatgtctggatctccccagcatgcctgctattctcttcccaatcctccccct
tgctgtcctgccccaccccaccccccagaatagaatgacacctactcagacaatgcgatgcaatttcctcattttattaggaa
aggacagtgggagtggcaccttccagggtcaaggaaggcacgggggaggggcaaacaacagatggctggcaactaga
aggcacag
SEQ ID NO: 2
aacttgtttattgcagcttataatggttacaaataaagcaatagcatcacaaatttcacaaataaagcatttttttcactgcattcta
gttgtggtttgtccaaactcatcaatgtatcttatcatgtctggatcCGTTACATAACTTACGGTAAATGG
CCCGCCTGGCTGACCGCCCAACGACCCCCGCCCATTGACGTCAATAATGAC
GTATGTTCCCATAGTAACGCCAATAGGGACTTTCCATTGACGTCAATGGGTG
GAGTATTTACGGTAAACTGCCCACTTGGCAGTACATCAAGTGTATCATATGC
CAAGTACGCCCCCTATTGACGTCAATGACGGTAAATGGCCCGCCTGGCATT
ATGCCCAGTACATGACCTTATGGGACTTTCCTACTTGGCAGTACATCTACGT
ATTAGTCATCGCTATTACCATGGTGATGCGGTTTTGGCAGTACATCAATGGG
CGTGGATAGCGGTTTGACTCACGGGGATTTCCAAGTCTCCACCCCATTGAC
GTCAATGGGAGTTTGTTTTGGCACCAAAATCAACGGGACTTTCCAAAATGT
CGTAACAACTCCGCCCCATTGACGCAAATGGGCGGTAGGCGTGTACGGTGG
GAGGTCTATATAAGCAGAGCTctctggctaactaccggtgccaccatggccccaaagaagaagcggaa
ggtcggtatccacggagtcccagcagccaagcggaactacatcctgggcctggacatcggcatcaccagcgtgggctac
ggcatcatcgactacgagacacgggacgtgatcgatgccggcgtgcggctgttcaaagaggccaacgtggaaaacaac
gagggcaggcggagcaagagaggcgccagaaggctgaagcggcggaggcggcatagaatccagagagtgaagaag
ctgctgttcgactacaacctgctgaccgaccacagcgagctgagcggcatcaacccctacgaggccagagtgaagggcc
tgagccagaagctgagcgaggaagagttctctgccgccctgctgcacctggccaagagaagaggcgtgcacaacgtga
acgaggtggaagaggacaccggcaacgagctgtccaccaaagagcagatcagccggaacagcaaggccctggaaga
gaaatacgtggccgaactgcagctggaacggctgaagaaagacggcgaagtgcggggcagcatcaacagattcaagac
cagcgactacgtgaaagaagccaaacagctgctgaaggtgcagaaggcctaccaccagctggaccagagcttcatcgac
acctacatcgacctgctggaaacccggcggacctactatgagggacctggcgagggcagccccttcggctggaaggac
atcaaagaatggtacgagatgctgatgggccactgcacctacttccccgaggaactgcggagcgtgaagtacgcctacaa
cgccgacctgtacaacgccctgaacgacctgaacaatctcgtgatcaccagggacgagaacgagaagctggaatattac
gagaagttccagatcatcgagaacgtgttcaagcagaagaagaagcccaccctgaagcagatcgccaaagaaatcctcgt
gaacgaagaggatattaagggctacagagtgaccagcaccggcaagcccgagttcaccaacctgaaggtgtaccacga
catcaaggacattaccgcccggaaagagattattgagaacgccgagctgctggatcagattgccaagatcctgaccatcta
ccagagcagcgaggacatccaggaagaactgaccaatctgaactccgagctgacccaggaagagatcgagcagatctct
aatctgaagggctataccggcacccacaacctgagcctgaaggccatcaacctgatcctggacgagctgtggcacaccaa
cgacaaccagatcgctatcttcaaccggctgaagctggtgcccaagaaggtggacctgtcccagcagaaagagatcccc
accaccctggtggacgacttcatcctgagccccgtcgtgaagagaagcttcatccagagcatcaaagtgatcaacgccatc
atcaagaagtacggcctgcccaacgacatcattatcgagctggcccgcgagaagaactccaaggacgcccagaaaatga
tcaacgagatgcagaagcggaaccggcagaccaacgagcggatcgaggaaatcatccggaccaccggcaaagagaa
cgccaagtacctgatcgagaagatcaagctgcacgacatgcaggaaggcaagtgcctgtacagcctggaagccatccct
ctggaagatctgctgaacaaccccttcaactatgaggtggaccacatcatccccagaagcgtgtccttcgacaacagcttca
acaacaaggtgctcgtgaagcaggaagaaaacagcaagaagggcaaccggaccccattccagtacctgagcagcagc
gacagcaagatcagctacgaaaccttcaagaagcacatcctgaatctggccaagggcaagggcagaatcagcaagacc
aagaaagagtatctgctggaagaacgggacatcaacaggttctccgtgcagaaagacttcatcaaccggaacctggtgga
taccagatacgccaccagaggcctgatgaacctgctgcggagctacttcagagtgaacaacctggacgtgaaagtgaagt
ccatcaatggcggcttcaccagctttctgcggcggaagtggaagtttaagaaagagcggaacaaggggtacaagcacca
cgccgaggacgccctgatcattgccaacgccgatttcatcttcaaagagtggaagaaactggacaaggccaaaaaagtga
tggaaaaccagatgttcgaggaaaagcaggccgagagcatgcccgagatcgaaaccgagcaggagtacaaagagatct
tcatcaccccccaccagatcaagcacattaaggacttcaaggactacaagtacagccaccgggtggacaagaagcctaat
agagagctgattaacgacaccctgtactccacccggaaggacgacaagggcaacaccctgatcgtgaacaatctgaacg
gcctgtacgacaaggacaatgacaagctgaaaaagctgatcaacaagagccccgaaaagctgctgatgtaccaccacga
cccccagacctaccagaaactgaagctgattatggaacagtacggcgacgagaagaatcccctgtacaagtactacgagg
aaaccgggaactacctgaccaagtactccaaaaaggacaacggccccgtgatcaagaagattaagtattacggcaacaaa
ctgaacgcccatctggacatcaccgacgactaccccaacagcagaaacaaggtcgtgaagctgtccctgaagccctaca
gattcgacgtgtacctggacaatggcgtgtacaagttcgtgaccgtgaagaatctggatgtgatcaaaaaagaaaactacta
cgaagtgaatagcaagtgctatgaggaagctaagaagctgaagaagatcagcaaccaggccgagtttatcgcctccttcta
caacaacgatctgatcaagatcaacggcgagctgtatagagtgatcggcgtgaacaacgacctgctgaaccggatcgaag
tgaacatgatcgacatcacctaccgcgagtacctggaaaacatgaacgacaagaggccccccaggatcattaagacaatc
gcctccaagacccagagcattaagaagtacagcacagacattctgggcaacctgtatgaagtgaaatctaagaagcaccct
cagatcatcaaaaagggcaaaaggccggcggccacgaaaaaggccggccaggcaaaaaagaaaaagTAGagaatt
cctagagctcgctgatcagcctcgactgtgccttctagttgccagccatctgttgtttgcccctcccccgtgccttccttgaccc
tggaaggtgccactcccactgtcctttcctaataaaatgaggaaattgcatcgcattgtctgagtaggtgtcattctattctggg
gggtggggtggggcaggacagcaagggggaggattgggaagagaatagcaggcatgctggggaggtaccgagggc
ctatttcccatgattccttcatatttgcatatacgatacaaggctgttagagagataattggaattaatttgactgtaaacacaaag
atattagtacaaaatacgtgacgtagaaagtaataatttcttgggtagtttgcagttttaaaattatgttttaaaatggactatcata
tgcttaccgtaacttgaaagtatttcgatttcttggctttatatatcttgtggaaaggacgaaacaccggagaccacggcaggt
ctcagttttagtactctggaaacagaatctactaaaacaaggcaaaatgccgtgtttatctcgtcaacttgttggcgagatttttg
cggcctccccagcatgcctgctattctcttcccaatcctcccccttgctgtcctgccccaccccaccccccagaatagaatga
cacctactcagacaatgcgatgcaatttcctcattttattaggaaaggacagtgggagtggcaccttccagggtcaaggaag
gcacgggggaggggcaaacaacagatggctggcaactagaaggcacag

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of integrating a heterologous polynucleotide into an endogenous gene in the genome of a cell, the method comprising:
a. administering to a cell a first recombinant nucleic acid comprising a heterologous polynucleotide comprising in 5′ to 3′ orientation a first terminator and a second terminator in reverse complement;
b. administering to the cell a second recombinant nucleic acid encoding a rare-cutting endonuclease targeted to a site within an endogenous gene in the genome of the cell and/or a gRNA sequence for targeting a rare-cutting endonuclease to a site within an endogenous gene in the genome of the cell; and
c. integrating the heterologous polynucleotide into the endogenous gene at the rare-cutting endonuclease target site to provide a modified endogenous gene in which the first terminator or the second terminator is operatively linked to a promoter of the endogenous gene;
wherein the modified endogenous gene produces an mRNA transcript that is truncated relative to an mRNA transcript produced by the endogenous gene.

2. The method of claim 1, wherein the first recombinant nucleic acid is a linear double-stranded or a linear single-stranded DNA molecule.

3. The method of claim 1, wherein the first recombinant nucleic acid is a circular double-stranded DNA molecule.

4. The method of claim 1, wherein the first recombinant nucleic acid is a viral vector.

5. The method of claim 4, wherein the viral vector is selected from the group consisting of an adenovirus vector, an adeno-associated virus vector, and a lentivirus vector.

6. The method of claim 5, wherein the viral vector is an adeno-associated virus vector.

7. The method of claim 1, wherein the rare-cutting endonuclease is selected from the group consisting of a zinc-finger nuclease, a meganuclease, a TALE nuclease, and a CRISPR nuclease.

8. The method of claim 1, wherein the first recombinant nucleic acid does not comprise a coding sequence and a coding sequence reverse complement operably linked to the first and second terminators.

9. The method of claim 3, wherein the first recombinant nucleic acid further comprises a rare-cutting endonuclease target site 5′ of the first and second terminators.

10. The method of claim 9, wherein the rare-cutting endonuclease target site within the recombinant nucleic acid is the same target site as within the endogenous gene.

11. The method of claim 1, wherein the endogenous gene is selected from DMPK, ATXN8, ATXN8OS, and JPH3.

12. The method of claim 11, wherein the first recombinant nucleic acid is integrated into the 3′ untranslated region of the DMPK gene.

13. The method of claim 12, wherein the first recombinant nucleic acid is integrated into the 3′ untranslated region of the DMPK gene downstream of the stop codon and upstream of the CTG repeat sequence.

14. The method of claim 1, wherein the recombinant nucleic acid further comprises left and right homology arms flanking the first and second terminators.

15. A method of integrating a heterologous polynucleotide into an endogenous gene in the genome of a cell, the method comprising:
a. administering to a cell a recombinant nucleic acid comprising a heterologous polynucleotide comprising in 5′ to 3′ orientation a first terminator, a sequence encoding a rare-cutting endonuclease targeted to a site within an endogenous gene in the genome of the cell, and a second terminator in reverse complement;
b. integrating the heterologous polynucleotide into the endogenous gene at the rare-cutting endonuclease target site to provide a modified endogenous gene in which the first terminator or the second terminator is operatively linked to a promoter of the endogenous gene;
wherein the modified endogenous gene produces an mRNA transcript that is truncated relative to an mRNA transcript produced by the endogenous gene.

16. The method of claim 15, wherein the recombinant nucleic acid is a linear double-stranded or a linear single-stranded DNA molecule.

17. The method of claim 15, wherein the recombinant nucleic acid is a circular double-stranded DNA molecule.

18. The method of claim 15, wherein the recombinant nucleic acid is a viral vector.

19. The method of claim 18, wherein the viral vector is selected from the group consisting of an adenovirus vector, an adeno-associated virus vector, and a lentivirus vector.

20. The method of claim 19, wherein the viral vector is an adeno-associated virus vector.

21. The method of claim 15, wherein the rare-cutting endonuclease is selected from the group consisting of a zinc-finger nuclease, a meganuclease, a TALE nuclease, and a CRISPR nuclease.

22. The method of claim 15, wherein the recombinant nucleic acid does not comprise a coding sequence and a coding sequence reverse complement operably linked to the first and second terminators.

23. The method of claim 17, wherein the recombinant nucleic acid further comprises a rare-cutting endonuclease target site 5′ of the first and second terminators.

24. The method of claim 23, wherein the rare-cutting endonuclease target site within the recombinant nucleic acid is the same target site as within the endogenous gene.

25. The method of claim 15, wherein the endogenous gene is selected from DMPK, ATXN8, ATXN8OS, and JPH3.

26. The method of claim 25, wherein the recombinant nucleic acid is integrated into the 3′ untranslated region of the DMPK gene.

27. The method of claim 26, wherein the first recombinant nucleic acid is integrated into the 3′ untranslated region of the DMPK gene downstream of the stop codon and upstream of the CTG repeat sequence.

28. The method of claim 15, wherein the recombinant nucleic acid further comprises left and right homology arms flanking the first and second terminators.

29. A polynucleotide comprising a first and second terminator in a tail-to-tail orientation, wherein the polynucleotide does not comprise a coding sequence operably linked to the first terminator and does not comprise a coding sequence operably linked to the second terminator.

30. The polynucleotide of claim 29, wherein the polynucleotide is a linear double-stranded or a linear single-stranded DNA molecule.

31. The polynucleotide of claim 30, wherein the polynucleotide is a circular double-stranded DNA molecule.

32. A recombinant viral vector comprising the polynucleotide of claim 29.

33. The recombinant viral vector of claim 32, wherein the viral vector is selected from the group consisting of an adenovirus vector, an adeno-associated virus vector, and a lentivirus vector.

34. The recombinant viral vector of claim 33, wherein the viral vector is an adeno-associated virus vector.

35. The polynucleotide of claim 29, further comprising a sequence encoding a rare-cutting endonuclease.

36. The polynucleotide of claim 29, further comprising an shRNA silencing cassette.

37. The polynucleotide of claim 35, wherein the rare-cutting endonuclease is selected from a the group consisting of a zinc-finger nuclease, a meganuclease, a TALE nuclease, and a CRISPR nuclease.

38. The polynucleotide of claim 31, further comprising a rare-cutting endonuclease target site 5′ of the first and second terminator.

39. The polynucleotide of claim 29, further comprising a left and right homology arm flanking the first and second terminators.

40. A method of integrating a heterologous polynucleotide into an endogenous gene in the genome of a cell, the method comprising:
a. administering to a cell a recombinant nucleic acid comprising a heterologous polynucleotide comprising in 5′ to 3′ orientation a first terminator, an shRNA silencing cassette, and a second terminator in reverse complement; and
b. integrating the heterologous polynucleotide into the endogenous gene at the rare-cutting endonuclease target site to provide a modified endogenous gene in which the first terminator or the second terminator is operatively linked to a promoter of the endogenous gene;
wherein the modified endogenous gene produces an mRNA transcript that is truncated relative to an mRNA transcript produced by the endogenous gene.
